(19) United States
(12) Patent Application Publication
(10) Pub. No.: US 2026/0334983 A1
(43) Pub. Date: Oct. 29, 2026

(54) SPINAL IMPLANT SYSTEMS WITH REVISION FEATURES

(71) Applicant: Nexus Spine, LLC, Salt Lake City, UT (US)

(72) Inventors: Peter A. Halverson, Salt Lake City, UT (US); David T. Hawkes, Salt Lake City, UT (US)

(73) Assignee: Nexus Spine, LLC, Salt Lake City, UT (US)

(21) Appl. No.: 19/738,218

(22) Filed: Jul. 10, 2026

Related U.S. Application Data
(63) Continuation-in-part of application No. 18/727,569, filed on Feb. 16, 2017.
(60) Provisional application No. 62/296,072, filed on Feb. 16, 2016.
(60) Provisional application No. 63/842,553, filed on Jul. 11, 2025.
(60) Provisional application No. 63/987,450, filed on Feb. 20, 2026.

(51) Int. Cl. A61B 17/70; A61B 17/00

(52) U.S. Cl. CPC A61B 17/7032; A61B 2017/00477

(57) ABSTRACT

Implementations of the disclosed systems and methods include one or more coupling assemblies for use in spinal fusion procedures. In some cases, the coupling assembly includes one or more: anchors configured to couple to spinal vertebrae; connectors configured to connect multiple anchors together; and couplers having one or more anchor receptacles for receiving the anchors and one or more connector receptacles for receiving the connector. In some cases, one or more collets are included as an adapter between the connector and the connector receptacle. Some implementations include one or more retention grooves formed within the anchor receptacle for better retention of the anchor. Other implementations are described.

### CROSS-REFERENCE TO RELATED APPLICATIONS

This application is a continuation-in-part of U.S. Patent Application No. 18/727,569 (Attorney Docket No. 23845.183), filed Jul. 9, 2024, and entitled “REVISION SYSTEM FOR SPINAL IMPLANTS,” which is a national stage entry under 35 U.S.C. § 371 of International Patent Application No. PCT/US2017/018244 (Attorney Docket No. 23845.78), filed Feb. 16, 2017, and entitled “REVISION SYSTEM FOR SPINAL IMPLANTS,” which claims the benefit of and priority to U.S. Provisional Patent Application No. 62/296,072 (Attorney Docket No. 23845.68), filed Feb. 16, 2016, and entitled “REVISION SYSTEM FOR SPINAL IMPLANTS.” This application also claims the benefit of and priority to U.S. Provisional Patent Application No. 63/842,553 (Attorney Docket No. 23845.190), filed Jul. 11, 2025, and entitled “SPINAL IMPLANT SYSTEM WITH REVISION FEATURES” as well as the benefit of and priority to U.S. Provisional Patent Application No. 63/987,450 (Attorney Docket No. 23845.260), filed Feb. 20, 2026, and entitled “SPINAL IMPLANT SYSTEM WITH REVISION FEATURES.” The disclosures of each of the foregoing applications are incorporated herein by reference in their entireties.

### FIELD

The present disclosure relates to spinal implants, and more particularly to systems for coupling rods and other spinal implant components, in some cases facilitating revision of prior spinal surgeries.

### BACKGROUND

Traditional spinal fusion surgery can be difficult and cumbersome, and it can also tend to trigger a cascade of adjacent spinal segment degeneration. The levels above and/or below a fused level can become painfully symptomatic within a few years. Subsequent fusion of an adjacent level can be difficult because the already-fused level still needs hardware to support the relatively weak bone of the fusion mass. However, some types of already-implanted hardware of traditional constructs preclude the implantation of new hardware at the adjacent level. Thus, historically, some subsequent surgeries required removal of present hardware and replacement with a longer implant construct. Such surgical procedures, however, can be risky and onerous, as removing an existing implant could be avoided. Also, removal surgery can cause complications, put weakened bone and other physiological structures at risk, and cause unnecessary damage and pain to patients.

Thus, while techniques currently exist that are used to revise previous spinal surgeries, challenges still exist, including those listed above. Accordingly, it would be an improvement in the art to augment or even replace current techniques with other techniques.

### SUMMARY

According to some implementations of the systems and methods provided herein, improved spinal constructs and methods are provided for implanting such constructs so as to allow for quicker and easier implantation, better outcomes, longer-lasting implants, and easier revision. In some cases, the systems and methods are configured to be used with either other improved implant constructs (in accordance with the present disclosure) or traditional implant constructs (which do not have some or all of the features of the improved constructs)—thus allowing for greater versatility and use in connection with a broader selection of patients.

In some implementations, one or more coupling assemblies are provided. The coupling assembly includes, in some implementations, one or more anchors configured to be secured to spinal vertebrae, one or more connectors configured to span between anchors, and one or more couplers configured to couple the anchors to the connectors. In some cases, the anchors include one or more shafts configured for implantation into bone and heads configured for coupling to the couplers, in some cases allowing polyaxial positioning prior to locking. In some implementations, the connectors include one or more rods or other elongated members having selectable lengths, diameters, and curvatures suitable for various spinal applications (e.g., cervical, thoracic, lumbar, sacral, or combinations of the foregoing).

In some implementations, the coupling assembly includes one or more collets configured to attach to an end of a connector and to interface with a connector receptacle of a coupler. The collet may be secured to the connector by deformation, compression, pinning, expansion, capping, or other retention mechanisms, and in some cases effectively extends the usable length of the connector. In some iterations, the collet-coupler interface permits polyaxial adjustment prior to selective locking.

In some implementations, the coupler includes an anchor receptacle configured to receive and retain an anchor head, and a connector receptacle configured to receive the connector or a collet coupled to the connector. The anchor receptacle may include one or more internal retention features, such as a groove formed in an inner surface, configured to increase retention strength while permitting polyaxial orientation of the anchor prior to locking. The connector receptacle of some iterations is selectively compressible and may be locked by a movable locking element, such that shifting the locking element secures the connector or collet within the coupler.

In some implementations, the disclosed systems are particularly well suited for revision procedures in which an existing spinal construct remains implanted. A new anchor may be placed at an adjacent vertebral level, a collet may be attached to an exposed end of an existing connector, and a coupler may be installed to join the new anchor to the existing construct without removing the original coupler or connector. This approach allows modular extension of a spinal construct while avoiding exposure and removal of prior implants. That said, some embodiments of the disclosed systems are also particularly well suited for initial procedures, while leaving the door open for later revision surgeries, should such become necessary.

In some implementations, the disclosed systems are particularly well suited for extension to an adjacent level of an existing construct. The extension construct may be attached in any suitable manner, including in-line with the existing construct but with a polyaxial joint to better allow for accommodating the varying anatomy along the patients’ spines. The disclosed systems are, in accordance with some implementations, not only polyaxial and attachable at end-of-construct, but they are also exceptionally low bulk and are also simple to employ.

The disclosure further provides methods for installing and removing coupling assemblies. In some implementations, one or more methods include preparing an existing implant, selecting and attaching a collet, placing a new anchor, selecting and installing a coupler, and locking the coupler to form a unified construct. Corresponding removal methods may include unlocking the coupler and disengaging the connector and anchor. Kits including anchors, connectors, collets, couplers, and associated instruments in multiple sizes and configurations are also provided to support these procedures.

Together, the disclosed systems and methods provide versatile, modular spinal implant solutions that improve ease of implantation, enable efficient revision and extension of existing constructs, and reduce the need for extensive hardware removal or replacement.

### DESCRIPTION OF THE FIGURES

The objects and features of the present disclosure will become more fully apparent from the following description and appended claims, taken in conjunction with the accompanying figures. Understanding that these figures depict only some embodiments of the disclosed systems and methods and are, therefore, not to be considered limiting in scope, the systems and methods will be described and explained with additional specificity and detail through the use of the accompanying figures in which:

FIG. 1A shows a side-perspective view of components of a coupling assembly, in accordance with some representative embodiments of the present systems and methods;

FIG. 1B shows a side-perspective view of some components of a coupling assembly, in accordance with some representative embodiments;

FIG. 1C shows a side-perspective view of some components of a coupling assembly, in accordance with some representative embodiments;

FIG. 1D shows a cross-sectional view of some components of a coupling assembly, in accordance with some representative embodiments;

FIG. 1E shows a side view of some components of a coupling assembly, in accordance with some representative embodiments;

FIG. 1F shows a cross-sectional view of some components of a coupling assembly, in accordance with some representative embodiments;

FIG. 1G shows a side perspective view of some components of a coupling assembly, in accordance with some representative embodiments;

FIGS. 2A-2C show side perspective views of some components of coupling assemblies, in accordance with some representative embodiments;

FIG. 3A shows a side perspective view of a collet on a connector, in accordance with some representative embodiments;

FIG. 3B shows a cross-sectional view of a collet on a connector, in accordance with some representative embodiments;

FIG. 3C shows a side perspective view of a collet on a connector, in accordance with some representative embodiments;

FIGS. 4A-4H show side perspective views of collets on connectors, in accordance with some representative embodiments;

FIGS. 5A-5C show side perspective views of some components of coupling assemblies, in accordance with some representative embodiments;

FIG. 6A shows a side perspective view of a coupler in an unlocked configuration, in accordance with some representative embodiments;

FIGS. 6B-6C show top perspective views of couplers in unlocked configurations, in accordance with some representative embodiments;

FIG. 6D shows a side perspective view of a coupler in a locked configuration, in accordance with some representative embodiments;

FIG. 7A shows a cross-sectional view of a coupler, in accordance with some representative embodiments;

FIG. 7B shows a perspective cross-sectional view of a coupler, in accordance with some representative embodiments;

FIG. 7C shows a cross-sectional view of a coupler as used in connection with a coupling assembly, in accordance with some representative embodiments;

FIG. 7D shows a perspective cross-sectional view of a coupler as used in connection with a coupling assembly, in accordance with some representative embodiments;

FIG. 8A shows a bottom perspective view of some components of a coupling assembly, in accordance with some representative embodiments;

FIG. 8B shows a plan view of some components of a coupling assembly, in accordance with some representative embodiments;

FIG. 8C shows a cross-sectional side view of some components of a coupling assembly, in accordance with some representative embodiments;

FIG. 9A shows a side perspective view of some components of a coupling assembly, in accordance with some representative embodiments;

FIG. 9B shows a top perspective view of some components of a coupling assembly, in accordance with some representative embodiments;

FIG. 9C shows a cross-sectional view of some components of a coupling assembly, in accordance with some representative embodiments;

FIG. 10A shows a bottom perspective view of a coupler, in accordance with some representative embodiments;

FIG. 10B shows a cross-sectional side view of a coupler, in accordance with some representative embodiments;

FIG. 11 shows a method for installing a coupling assembly, in accordance with some representative embodiments;

FIGS. 12A-12N show illustrative portions of some methods for installing a coupling assembly, in accordance with some representative embodiments;

FIG. 13 shows a method for removing a coupling assembly, in accordance with some representative embodiments;

FIGS. 14A-14D show illustrative portions of some methods for removing a coupling assembly, in accordance with some representative embodiments;

FIGS. 15A-15B show side perspective views of anchors, in accordance with some embodiments;

FIGS. 15C-15D show X-ray illustrations of anchors as used in connection with spinal vertebrae, in accordance with some embodiments;

FIG. 16 shows a side perspective view of a collet, in accordance with some embodiments;

FIG. 17 shows a side perspective view of a prior-art revision system;

FIGS. 18A-20 show various views of coupling assemblies, in accordance with some embodiments; and

FIGS. 21A-21D show side perspective views of tulip-based coupling assemblies, in accordance with some embodiments.

### DETAILED DESCRIPTION

A description of embodiments will now be given with reference to the figures. It is expected that the present systems and methods may take many other forms and shapes. Hence, the following disclosure is intended to be illustrative and not limiting, and the scope of the disclosure should be determined by reference to the appended claims.

According to some embodiments, one or more spinal implant assemblies, or coupling assemblies 100, are provided. Generally speaking, the coupling assembly can include any suitable component configured to aid or assist in a spinal operation, such as spinal fusion surgery. Indeed, the coupling assembly can include any component suitable for coupling multiple spinal vertebrae of a patient together, or for applying any desired forces, reinforcement, adjustment, structure, or other desirable elements of a spinal procedure to the patient’s spine. For example, some embodiments of the coupling assembly include one or more: anchors 102 (e.g., for implanting into a bone or another portion of a patient); connectors 120 (e.g., for connecting the anchors together or providing a specific shape or structure to influence the bones in which the anchors are implanted); or couplers 150 (e.g., for coupling the anchors to the connectors). Each of these components in turn can include any suitable feature or component, as described in more detail below.

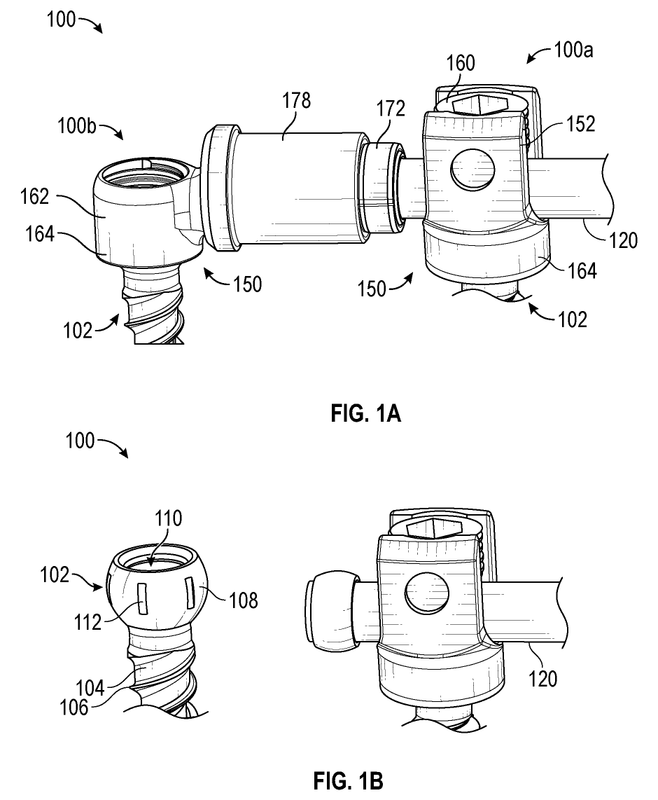

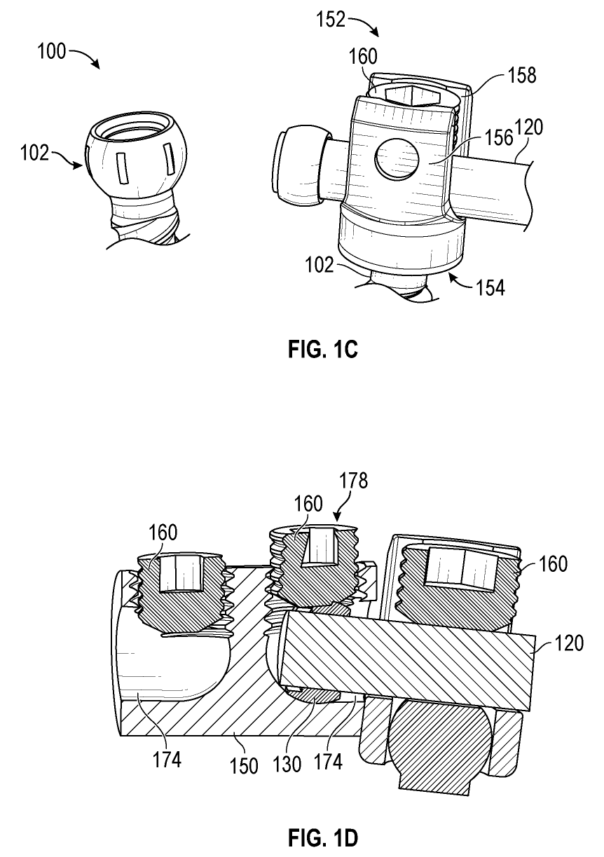

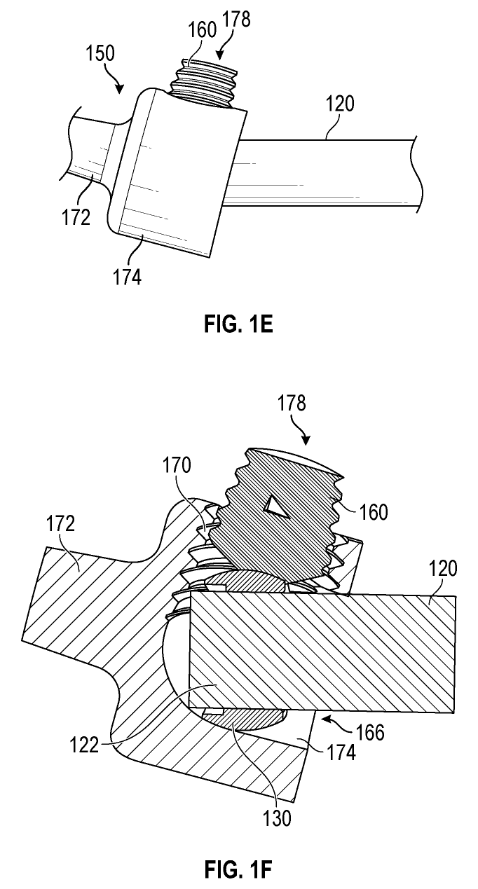

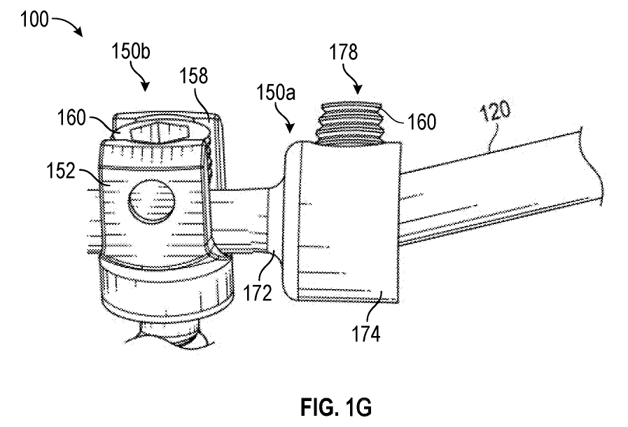

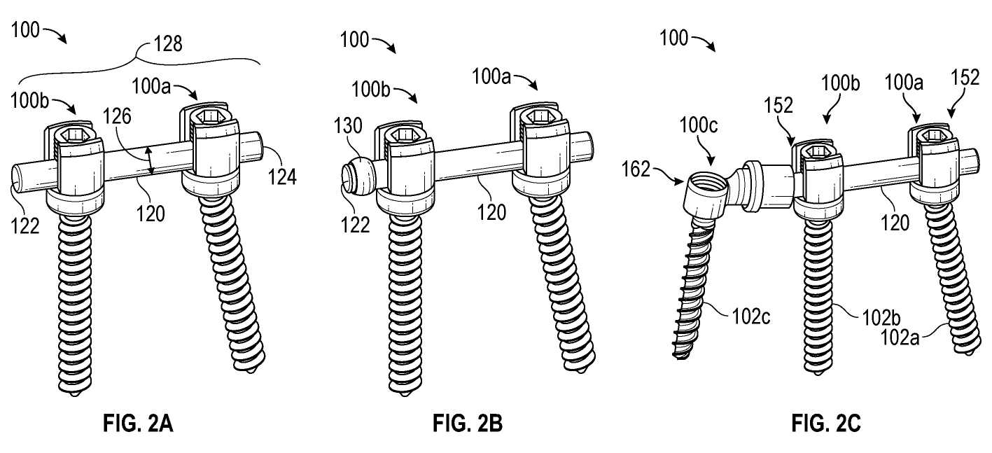

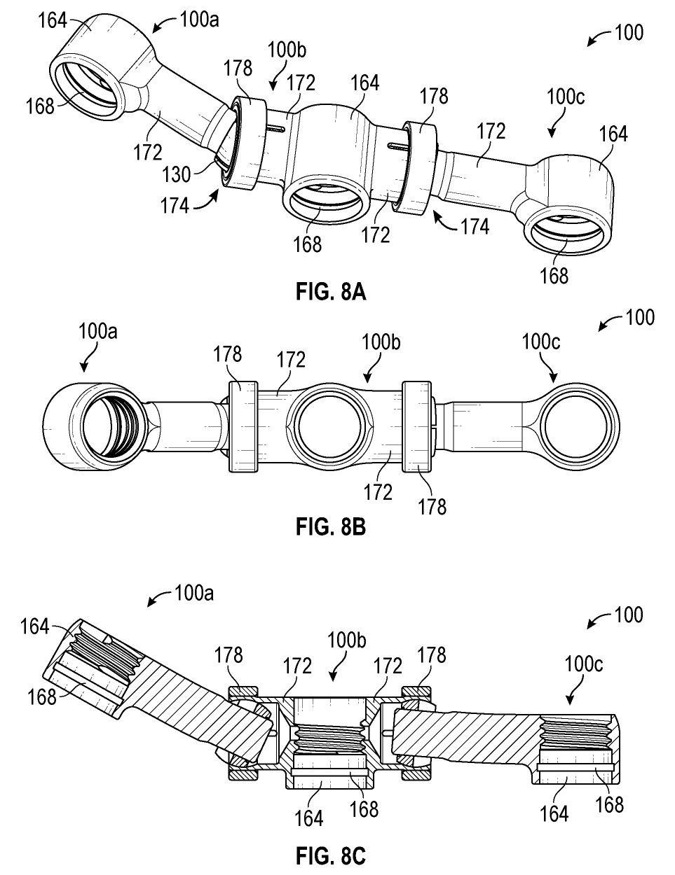

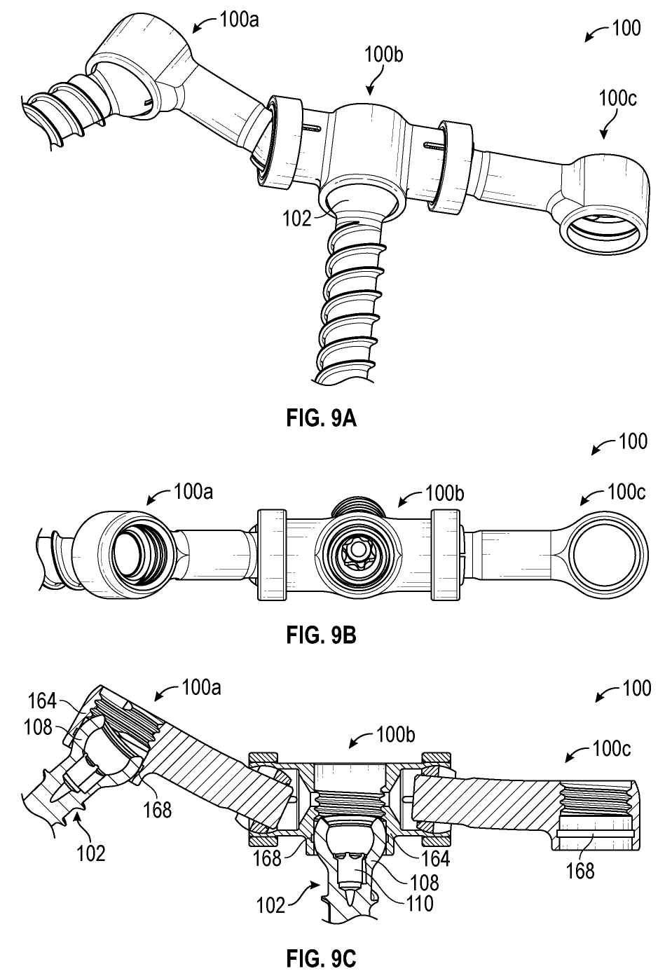

According to some embodiments, the coupling assembly 100 includes one or more first portions 100a, one or more second portions 100b, one or more third portions 100c, and any number of additional portions. Although the portions can include any suitable parts of the implant, some embodiments of the first portion are configured to couple to a first vertebra of a patient’s spine, some embodiments of the second portion are configured to couple to a second vertebra of the patient’s spine, some embodiments of the third portion are configured to couple to a third vertebra of the patient’s spine, and so on. In some cases, one portion (e.g., the first portion, the second portion, or another portion) comprises an original portion (e.g., installed in accordance with a first procedure), and another portion (e.g., the second portion, the third portion, or another portion) comprises a revision portion (e.g., installed in accordance with a later revision procedure). In some cases, multiple portions are installed in a single procedure. By way of non-limiting illustration, FIG. 1A shows some embodiments of a coupling assembly 100 having a first portion 100a and a second portion 100b, where the first portion is configured to couple to a first vertebra (not shown), and the second portion is configured to couple to a second vertebra (not shown), wherein the first portion 100a may have been installed in an earlier procedure, or it may be installed concurrently with the second portion 100b. Similarly, FIGS. 2A-C show some embodiments of coupling assemblies 100 with first portions 100a and second portions 100b, with FIG. 2C further showing some embodiments comprising an additional third portion 100c (which may be a revision portion, or which may be implanted concurrently with the first portion 100a and the second portion 100b). By way of further illustration, FIGS. 8A-9C show some embodiments of coupling assemblies 100 with first portions 100a configured to couple to a first vertebra, second portions 100b configured to couple to a second vertebra, and third portions 100c configured to couple to a third vertebra.

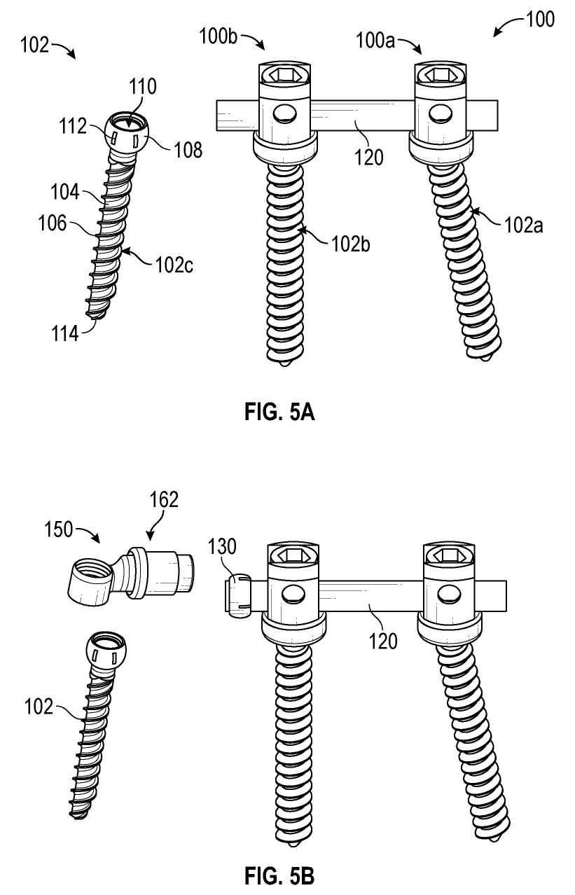

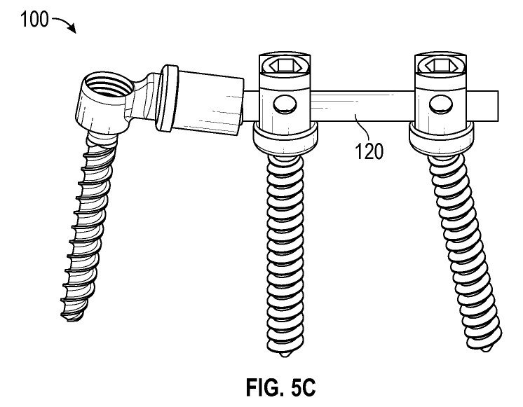

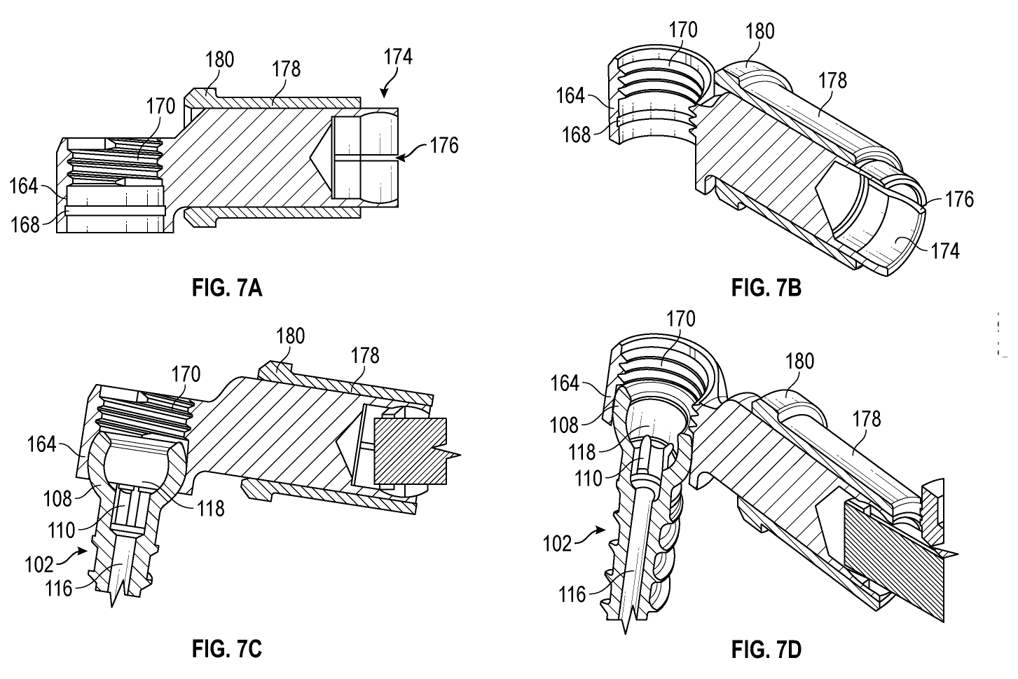

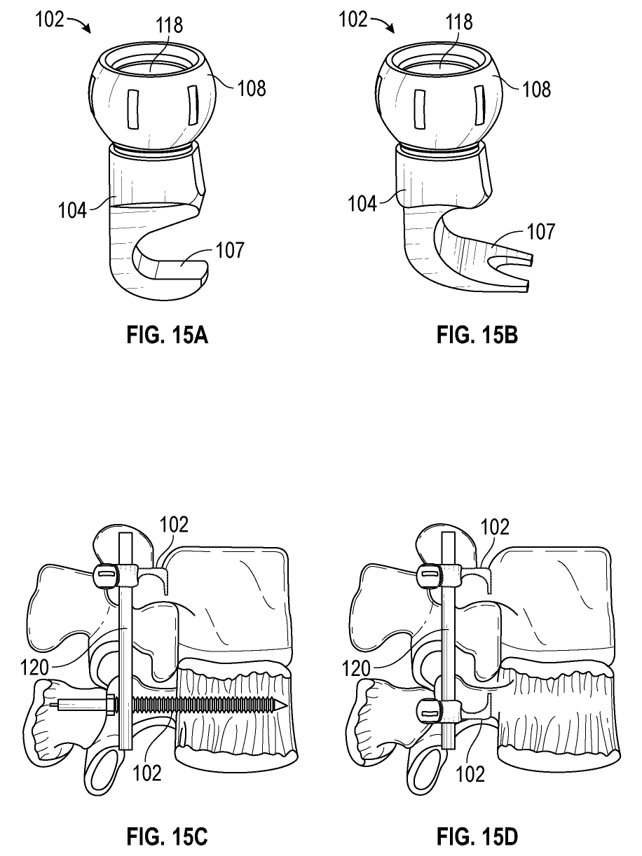

As mentioned above, some embodiments of the coupling assembly 100 include one or more anchors 102. The anchors can include any suitable components for allowing the anchors to couple (directly or indirectly) to one or more bones (e.g., vertebrae or other bones) or other portions of a patient (e.g., muscles, tissues, ligaments, or other parts of the patient). For example, some embodiments of the anchors include one or more pedicle screws, bone screws, nails, bolts, pins, rods, staples, plates, or any other suitable anchors. Some embodiments of the anchors include one or more shafts 104, threadings (or threaded portions) 106, hooks (or hooked portions) 107, heads 108, drives 110, retention features 112, tips 114, channels 116, or any other suitable components. Additionally, the coupling assembly can include any suitable number of anchors, such as a first anchor 102a, a second anchor 102b, a third anchor 102c, and any additional number of anchors. In some cases, the first anchor 102a is associated with the first portion 100a of the coupling assembly, the second anchor 102b is associated with the second portion, the third anchor 102c is associated with the third portion, and so on. By way of non-limiting illustration, FIGS. 1B-1C show examples of coupling assemblies 100 with anchors 102 (with some components of the coupling assemblies 100 not shown so that the anchors 102 are more visible). FIG. 2C shows an example of a coupling assembly 100 with a first anchor 102a, a second anchor 102b, and a third anchor 102c. Cross sections of anchor 102, in accordance with some embodiments, can be seen in FIGS. 7C-7D.

Where the anchor 102 includes a shaft 104, any suitable shaft for allowing the anchor to be embedded in a bone (or any other suitable portion of a patient) can be used. For example, some embodiments of the shaft are threaded (i.e., they include a threading 106); some are smooth, textured, or otherwise unthreaded; some are barbed or hooked (e.g., they include a hook 107); and others comprise a combination of the foregoing. The shaft can also have any suitable (outer) diameter (e.g., between about 2 mm and 12 mm, or any other suitable diameter as used in pedicle screws or other anchors) and any suitable length (e.g., between about 10 mm and 90 mm, or any other suitable length as used in pedicle screws or other anchors). In some cases, various anchors of different sizes are provided as desirable for different patients or different portions of the spine. As some non-limiting examples, some anchors include 5.0 x 35 mm screws, 6.5 x 45 mm screws, 4.5 x 50 mm screws, or any other suitable anchor sizes. The shaft can have any suitable shape, such as by being tapered, generally cylindrical, hollow, coiled, or any other suitable shape.

Where the shaft 104 includes threading 106, the threading can include any suitable threading, with any suitable size, pitch, threads per cm, number of starts, profile, thread form, thread angle, crest shape, root shape, thread direction (hand), surface finish, coating, or any other suitable features. For example, some embodiments of the threading have a major (outer) diameter corresponding generally to the shaft diameter and a minor (core) diameter that is smaller by about 1 mm to about 3 mm. In some embodiments, the threading includes a coarse pitch, such as between about 2 mm and 4 mm per thread, and in some embodiments, the threading includes a finer pitch, such as between about 1 mm and 2 mm per thread. Some embodiments of the threading include one or more additional features, such as a variable pitch, a dual-lead thread, a buttress thread profile, a V-shaped thread profile, a square or modified square thread profile, or any other suitable threading feature. The threading of some embodiments extends along substantially the entire length of the shaft, and the threading of some embodiments extends along only a portion of the shaft (e.g., 5%, 10 %, 25%, 40%, 50%, 75%, 80%, 90%, or any other suitable portion, whether near the anchor head 108, near the tip 114, in the middle of the shaft, some combination of the foregoing, or otherwise). The threading can be self-tapping or non-self-tapping, and it can include one or more cutting flutes or reliefs.

Where the shaft 104 includes a hook 107, any suitable type of hook can be used, including one or more barbs, flanges, curves, angles, catches, single-tipped hooks, double-tipped hooks, hooks with other configurations, or other hooking portions configured to catch on or embed within a bone or another portion of a patient. By way of non-limiting illustration, FIGS. 15A-15B show examples of anchors 102 having shafts 104 with hooks 107. Further, FIGS. 15C-15D show examples of how anchors 102 with hooks may be used in connection with spinal vertebrae, in accordance with some embodiments.

Where the anchor 102 includes a head 108, the head can include any suitable component for interfacing with one or more tools, or with one or more other portions of the coupling assembly 100 (such as the coupler 150). For example, some embodiments of the head include one or more drives 110, retention features 112, external surfaces, or other interfacing components. Additionally, the head can have any suitable shape, such as by being generally spherical (e.g., rounded, having one or more generally spherical portions, etc.), cylindrical, cuboid, pyramidal, conical, prismatic, toroidal, polyhedral, faceted polyhedral, or otherwise shaped. That said, some embodiments are generally spherical in order to allow for a polyaxial coupling with the coupler or to interface with a retention groove 168 as described in more detail in a later portion of this specification. By way of non-limiting illustration, FIGS. 1B and 5A show anchors 102 having heads 108 with drives 110, retention features 112, and generally spherical or rounded shapes for polyaxial coupling prior to locking.

Where the anchor 102 has a head 108 with a drive 110, any suitable drive can be used. For example, some embodiments include one or more: hexalobular or hex drives, square drives, cruciform drives, Phillips drives, star-shaped drives, lobed drives, spline drives, flat drives, or any other suitable drive configuration for transmitting torque from a tool to the anchor. In some embodiments, the drive 110 is recessed within the head 108, while in some embodiments the drive 110 is formed on an exterior (or any other suitable) portion of the head 108. The drive 110 can be configured to accept a corresponding driver tool of any suitable size. By way of non-limiting illustration, FIGS. 7C-7D show anchors 102 having drives 110 recessed within anchor heads 108 (the anchor heads therefore defining a recess that is part of or that is fluidically coupled to the channel 116, as discussed below).

Where the anchor 102 includes a tip 114, the tip can have any suitable configuration. For example, in some embodiments, the threading 106 extends all the way to the end of the tip, or the tip includes self-drilling or self-tapping features.

Where the anchor 102 includes one or more channels 116, the channel can have any suitable configuration. For example, in some embodiments, the channel extends all the way through the anchor, from the drive 110 to the tip 114 to allow for fluids to pass through the anchor (for example, in some cases this allow blood or lymph to escape from one or more portions of the coupler assembly 100). In some embodiments, the channel has a diameter sufficiently wide to accommodate a K-wire or another guiding mechanism as may be threaded through the anchor to guide the anchor its proper location. In some cases, the channel includes one or more recesses 118 in the anchor head 108. In some cases, the recess is configured to receive a portion of a tool (e.g., a head of a wand). By way of non-limiting illustration, FIG. 5A shows some embodiments of an anchor 102 with a sharpened tip 114, and FIGS. 7C-7D show some embodiments of anchors 102 having channels 116 passing therethrough, the channels including or being contiguous with recesses 118 in the corresponding anchor heads 108.

In accordance with some embodiments, the coupling assembly 100 includes one or more connectors 120. In this regard, the connector can include any suitable components configured to link the implant across vertebrae or to connect two or more separate anchors 102 together. For example, the connector can include one or more rods or other connecting components, such as dowels, bolts, pipes, wires, beams, batons, shafts, bars, struts, or other such components (particularly as may be useful for spanning all or part of a distance between a first anchor 102a and a second anchor 102b). While some embodiments of the connector include circular rods (e.g., they have a substantially circular cross section), some embodiments include square rods, triangular rods, polygonal prismatic rods, other types of rods or other connectors (e.g., connectors having a cross section that is substantially semi-circular, triangular, square, rectangular, trapezoidal, pentagonal, hexagonal, star-shaped, T-shaped, plus-shaped, polygonal, elliptical, or any other regular or irregular shape). By way of non-limiting illustration, FIG. 2A shows a coupling assembly 100 having a connector 120 in the form of a rod with a substantially circular cross section.

According to some embodiments, the connector 120 includes one or more of a first end 122, a second end 124, a diameter 126 (which, in some embodiments, is generally constant throughout the rod, or which, in some embodiments, is variable so as to provide a rod with different diameters at different portions), and a length 128. For example, FIG. 2A shows some embodiments of a coupling assembly 100 with a connector 120 in the form of a rod having a first end 122, a second end 124, a diameter 126, and a length. For purposes of clarity, the term “diameter” is not limited to a circular diameter, but is also used herein to refer to a width or thickness of a connector with a non-circular cross section.

The connector 120 can have any suitable diameter 126 and length 128. For example, in some embodiments, the connector has a diameter of between about 2 mm and about 8 mm (or any subrange thereof), such as about 3.5 mm, 4.0 mm, 4.5 mm, 5.0 mm, 5.5 mm, 6.0 mm, or 6.35 mm, or any other suitable diameter suitable for use in connection with spinal fixation rods or other connectors. In some embodiments, smaller diameters are used (e.g., for cervical or thoracic applications), while larger diameters are used in some cases (e.g., for lumbar or sacral applications). The length of the connector can likewise vary depending on the intended anatomy and number of anchors to be coupled, but in some embodiments the length is between about 30 mm and about 60 cm (or any subrange thereof), such as between about 40 mm and 30 cm, between about 50 mm and 35 cm, or any other suitable length. Additionally, the connector can have any desired curvature, angle, bend, or other shape. For example, in some cases, a curvature is desired to allow the connector to generally conform to an anatomical curvature of a patient’s spine, such as cervical lordosis, thoracic kyphosis, or lumbar lordosis. In some embodiments, the connector is pre-curved to approximate a typical or average spinal curvature for a given spinal region, while in other embodiments the connector is substantially straight and is contoured intraoperatively by a surgeon to achieve a desired curvature. In still other embodiments, the connector includes one or more discrete curved segments, compound curves, or a continuously varying curvature along its length. The curvature can be uniform or non-uniform, symmetric or asymmetric, and can vary in magnitude along different portions of the connector. In some embodiments, the curvature is selected to facilitate reduction of one or more vertebrae, restoration or maintenance of sagittal or coronal alignment, correction of deformity, or distribution of loads across the coupling assembly. Some embodiments of the connector include one or more regions configured to resist or maintain curvature after contouring, or to permit elastic or plastic deformation during implantation.

In some embodiments, the connector 120 is provided in one or more straight lengths configured to be cut or contoured intraoperatively, and in some embodiments, the connector is pre-cut, pre-contoured, or pre-bent to a desired length or curvature. The connector can also include any other suitable feature to assist it in its function of joining multiple anchors 102 together. For example, some embodiments of the connector 120 include one or more regions of reduced diameter 126, increased diameter, surface texturing, processes, recesses, flattened portions, catches, mechanical engagements, or other features configured to facilitate engagement with a coupler 150 (as described in more detail below), a set screw, or another portion of the coupling assembly 100.

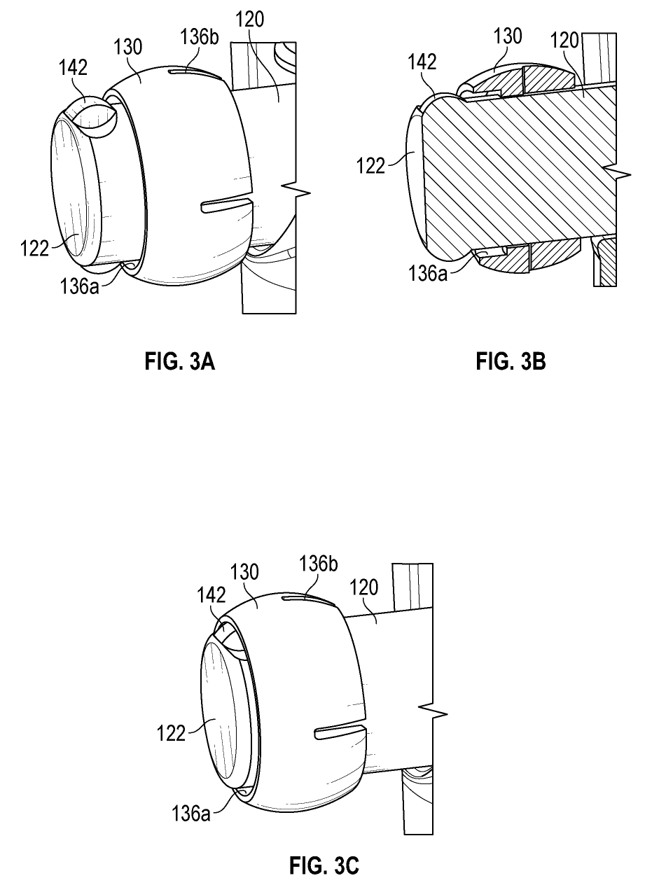

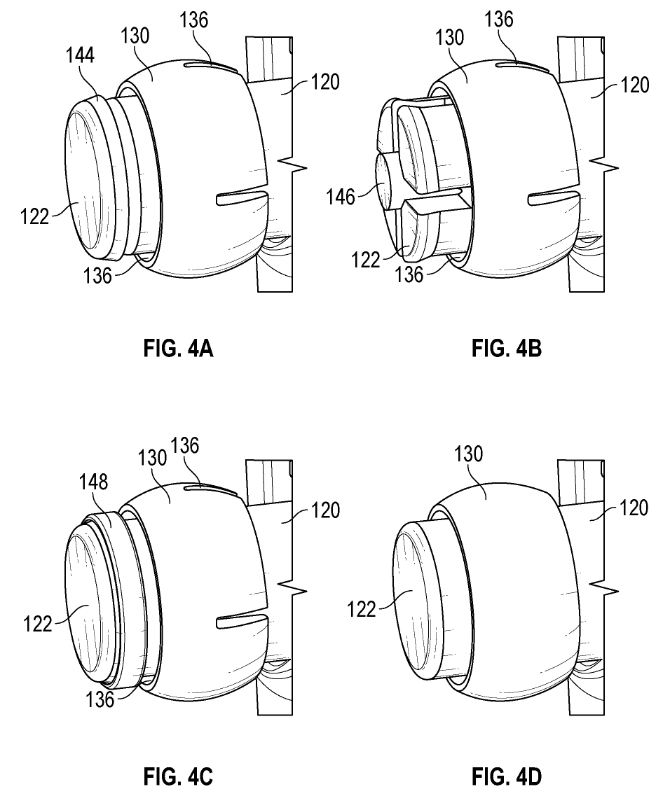

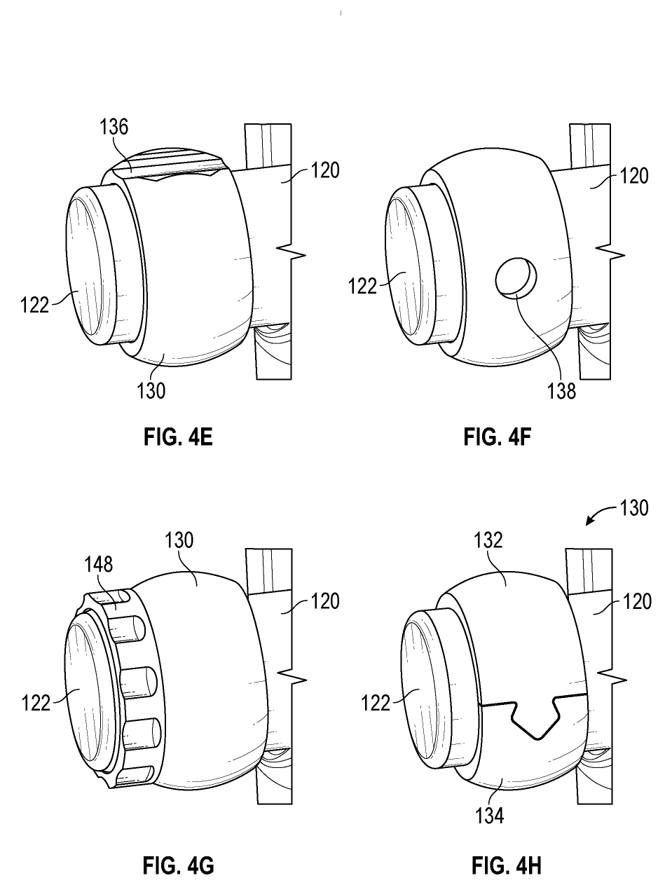

Some embodiments of the connector 120 are configured to extend past a coupler 150. For example, in some cases, the length 128 of the connector is greater than a distance between a first portion 100a and a second portion 100b of the coupling assembly (or a first anchor 102a and a second anchor 102b) to be joined together by the connector. Accordingly, in some cases, at least one of the first end 122 and the second end 124 of the connector extends past the associated coupler, such that the end in question is free to be used. In some embodiments, as described in additional detail below, the coupling assembly 100 includes a coupler configured to couple to the free end of the connector. To this end, some embodiments of the connector include one or more components configured to facilitate such a coupling, such as one or more collets 130 or one or more retention features 140.

Where the coupling assembly 100 includes one or more collets 130, the collet can include any suitable component configured to selectively, permanently, or semi-permanently attach to at least one of the first end 122 and the second end 124 of the connector to facilitate a coupling with a coupler 150 (as discussed in more detail below). Accordingly, the collet can include any component configured to be disposed around the diameter 126 of the connector, or to otherwise be clipped onto, hooked onto, slid onto, adhered to, wrapped around, passed through, formed on, or otherwise coupled to the connector (more particularly, to an end of the connector). Thus, although the collet 130 can have any suitable shape, some embodiments of the collet have an annular configuration (e.g., forming a toroidal, hollow-cylinder, ring, or other annular shape around the connector). Additionally, some embodiments of the collet have a rounded surface (e.g., similar to a rounded anchor head 108) to allow for a polyaxial connection with the coupler 150. For example, FIGS. 3A-4G show examples of collets 130 configured to be selectively slid or otherwise placed onto a first end 122 of a connector 120, and FIG. 4H shows an example of a collet 130 configured to selectively separate into either a first collet portion 132 and a second collet portion 134 configured to join together to form the collet 130 around the connector 120 or a belt that is configured to couple at its end. In each of these FIGS., the collet 130 has an annular configuration with a rounded surface after installation.

According to some embodiments, the collet 130 is configured to be secured in place (e.g., around the connector 120 after the collet has been disposed in a desirable position). Accordingly, some embodiments include one or more compression features 136, through-holes 138, or other features configured to allow the collet to be secured in place.

Where the collet 130 includes a compression feature 136, the compression feature can include any suitable feature that allows the collet or any portion thereof to be compressed, shrunk, warped, deformed, or otherwise modified such that it becomes secured around the connector 120 (and can no longer slide or move along the connector). For example, some embodiments of the compression feature include one or more: gaps 136a between the collet and the connector (such that a portion of the collet can be crimped or otherwise modified to collapse the gap and cause that portion of the collet to press against the connector, thereby creating a high-friction engagement); slits (such that portions of the collet can be pressed closer together, compressing the slits and causing a tighter fit); or other features configured to allow for compression or other modification for a tighter fit. In some cases, the collet (or a portion thereof) includes a deformable material configured to be crimped, thermoformed, heat-set, compressed, or otherwise deformed to cause the collet to more securely engage with the connector. By way of non-limiting illustration, FIGS. 3A-3C show some embodiments of collets 130 with compression features in the form of gaps 136a and slits 136b.

Where the collet 130 includes one or more through-holes 138, the through-hole can include any suitable passage to allow for a process (e.g., a process on the connector 120), a pin, or any other suitable locking element to be fully or partially disposed through the through-hole to secure the collet to the connector. In some cases, a corresponding hole on the connector is configured to align with the through-hole such that a locking element can be disposed through both the through-hole of the collet and the hole of the connector to lock the components into position with respect to each other. By way of non-limiting illustration, FIG. 4F shows some embodiments of a collet 130 with a through-hole 138.

According to some embodiments, instead of or in addition to a compression feature 136, through-hole 138, or other retaining element being included on or with the collet 130, the connector 120 includes one or more retention features 140. For examples, in some cases, a first end 122 or a second end 124 of the connector includes a retention feature that prevents the collet from sliding off the end or that holds the collet in place (accordingly, in some cases, the collet is still permitted to slide along the connector, but it is prevented from sliding off the end, and in some cases, the collet is fixed in place with respect to the connector).

Where a retention feature 140 is included, the retention feature can include any suitable feature for retaining the collet 130 on the connector 120. For example, some embodiments of the retention feature include one or more: deformable portions 142 (which, like some embodiments of the compression feature 136, are configured to be deformed or otherwise modified to retain the collet on the connector); rims 144 (e.g., a lip, flange, step, or other portion of the connector, which may be at the end of the connector or at any other suitable portion, with a larger diameter than another portion of the connector, such that the collet cannot slide off the end); expanders 146 (e.g., configured to be inserted into an end of the connector or otherwise expand the connector so that the collet cannot slide off); or caps 148 (e.g., components configured to be inserted onto or over an end of the connector to prevent the collet from sliding off). Where one or more caps are included, the caps can be configured to screw onto the end of the connector, be crimped or otherwise deformed to form a strong coupling with the connector, be glued onto the connector or otherwise attached with an adhesive, be welded in place, or otherwise be coupled to the connector to prevent (in some cases, selectively) the collet from disengaging with the connector.

By way of non-limiting illustration, FIGS. 3A-4H show some ways in which the collet 130 can be secured to the connector 120. In FIGS. 3A-3C, a deformable portion 142 of the connector 120 is (in accordance with some embodiments) deformed to prevent the collet 130 from sliding off. In FIG. 4A, a rim 144 is included (in some embodiments) on the end 122 of the connector 120 to prevent the collet 130 from sliding off. In FIG. 4B, an end 122 of the connector 120 is (in accordance with some implementations) divided to allow for the insertion of an expander 146, which causes the end to expand, thereby preventing the collet 130 from sliding off. In FIG. 4C, a cap 148 in the form of a ring is (in accordance with some cases) provided to prevent the collet 130 from sliding off. In FIGS. 4D-4E, the material of the collet 130 is deformable, such that, in some cases, the collet can be crimped to form a compression feature 136 that prevents the collet 130 from sliding off. In FIG. 4F, a through-hole 138 is provided such that a pin or another locking component can be inserted to prevent the collet 130 from moving with respect to the connector 120 in accordance with some embodiments. In FIG. 4G, a cap 148 in the form of a ring is provided to prevent the collet from sliding off some embodiments. In FIG. 4H, the collet 130 is (in accordance with some cases) provided in bifurcated form, with a first collet portion 132 and a second collet portion 134 configured to fit together strongly enough to form a tight fit with the connector 120 such that it is prevented from sliding off.

According to some embodiments, the end of the connector 120 does not extend far enough past the associated coupler 150 for the collet 130 to be slid fully onto the length of the connector. In some such cases, the collet is configured to slide only part-way onto the connector and still form a strong coupling with the connector, thereby effectively extending the length of the connector. Indeed, some embodiments of the collet include one or more necks 135, extensions, bases, sleeves, tubes, threadings, or other portions configured to allow the collet to more effectively extend the length of the connector past its associated coupler. For example, in some cases, a double collet is provided, with the collet having a first portion configured to couple to the connector (as described above) and a second portion configured to couple to a connector receptacle 174 (as discussed below). Also, in accordance with some embodiments, the collet 130 can be placed at varying distances from the end of the connector before being locked to the connector. Furthermore, some embodiments of the collet 130 can also be the end of another connector, such that an existing connector and a new connector are joined at the collet 130 and then crimped (or otherwise coupled) together at the collet 130 with any type of external compression device (or in any other suitable manner). In accordance with some embodiments, the collet 130 is configured to be crimped to the end of an existing construct by a set screw within a polyaxial coupler to extend an existing construct to new spinal levels.

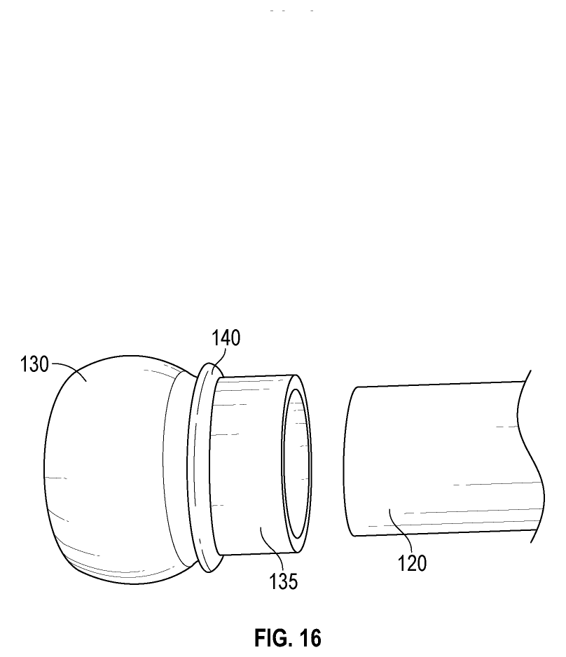

As another example of a collet 130 configured to extend the length of the associated connector 120, some embodiments of the collet include one or more necks 135. In some cases, the neck includes one or more openings that open to one or more passages configured to receive one or more connectors (e.g., in some cases, the neck is configured to receive the end of the associated connector). In some cases, the neck is slim enough not only to slide onto the connector, but to fit within a coupler 150 (such as a tulip coupler 152) that is receiving the connector, thereby allowing the collet to couple to a portion of the connector that is already disposed within the tulip (or other coupler). In some embodiments, the neck can slide over the end of the connector any suitable amount. For example, in some cases, only the end of the neck slides over the end of the connector, but in some cases, a larger portion of the end of the connector slides into the opening and is received within the neck. In some cases, the passage extends into the body of the collet, such that the end of the connector can selectively be disposed within the body of the collet.

Where the collet 130 includes a neck 135, the neck can be configured to couple to the connector 120 in any suitable manner. For example, in some cases (as discussed above), the neck is configured to receive the connector. That said, in some embodiments, the connector is configured to receive the neck or the neck is otherwise configured to couple to the connector. Additionally, as discussed above, some embodiments of the collet include one or more retention features 140 (for retaining the collet on the connector). In this regard, in some embodiments where the neck is configured to receive the connector (or vice versa), a retention feature in the form of a ring is included. In some cases, the ring is configured to compress a portion of the neck (e.g., near the opening), thereby forming a tight coupling between the neck and the connector. Indeed, the retention feature can include any feature of a locking element 178 as discussed in more detail below. By way of non-limiting illustration, FIG. 16 shows a collet 130 having a neck 135, the neck being configured to receive a connector 120, with a retention feature 140 in the form of a ring being disposed around the neck such that when the neck is disposed over the connector, the ring can be slid toward the opening of the neck, thereby compressing the portion of the neck in which the connector is disposed, thereby forming a tight fit between the connector and the neck.

In accordance with some embodiments, the coupling assembly 100 includes one or more couplers 150. For example, some embodiments include a first coupler 150a, a second coupler 150b, a third coupler 150c, and any number of additional couplers. Where a coupler is included, the coupler can include any component configured to couple the anchor 102 to the connector 120. As discussed in more detail below, couplers can include tulip couplers 152, press-on couplers 162, or any other suitable types of couplers.

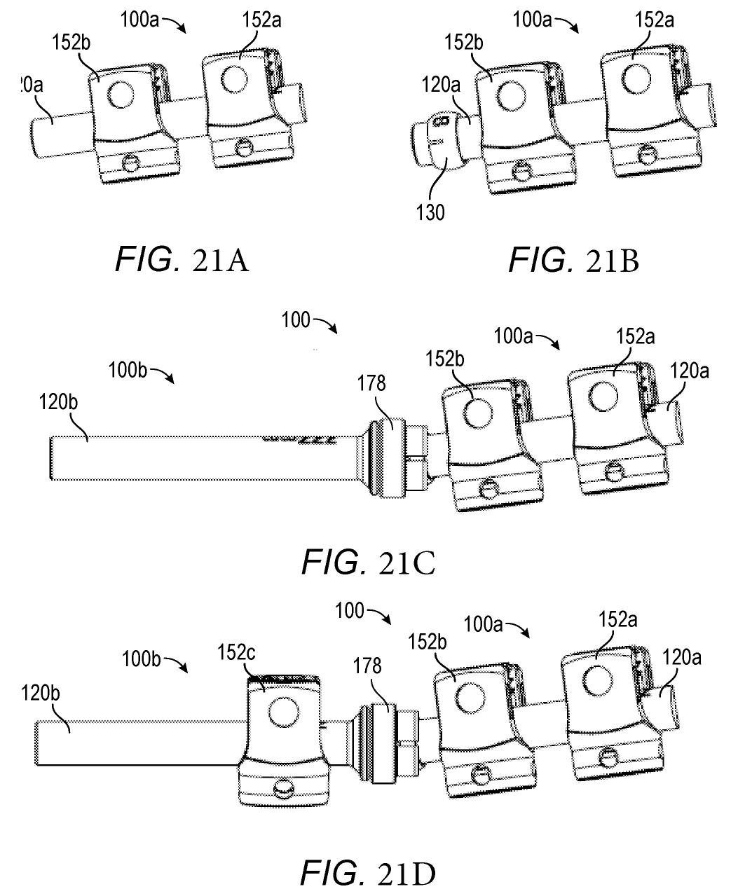

In some embodiments, one or more of the couplers 150 include tulip couplers 152. In some embodiments, the tulip coupler includes a bottom having an anchor hole 154 formed therein, and a top having a tulip 156 with a slot 158 formed in it. In some embodiments, the tulip coupler is configured such that an anchor 102 is configured to be inserted, shaft-first through the tulip and out the anchor hole, with the anchor head 108 not fitting through the anchor hole, such that the anchor head is retained within the tulip while the shaft 104 extends away from the bottom of the tulip out of the anchor hole. In some embodiments, a connector 120 is configured to be disposed within the slot (e.g., with a first end 122 of the connector extending away from the tulip in a first lateral direction, and a second end 124 of the connector extending away from the tulip in a second lateral direction, often generally opposite the first lateral direction). By way of non-limiting illustration, FIG. 1C shows a tulip coupler 152 having an anchor hole 154 formed in the bottom thereof with an anchor 102 extending out of it, the tulip coupler 152 further including a tulip 156 with a slot 158 formed therein, a connector 120 in the form of a rod disposed in the slot, and a set screw 160 inserted into the top of the tulip to prevent the connector from coming back out through the top. By way of further illustration, FIGS. 21A-21D show some embodiments of coupling assemblies 100 having a first tulip coupler 152a and a second tulip coupler 152b (on a first portion 100a of the coupling assembly 100), and (in the case of FIG. 21D) a third tulip coupler 152c on a second portion 100b of the coupling assembly 100. This further illustrates that the systems and methods described herein can be implemented using any suitable types of couplers 150.

As mentioned above, some embodiments of the coupling assembly 100 are particularly useful for revision procedures (although some embodiments are also particularly useful for initial procedures). With that in mind, it is worth noting that tulip couplers 152 have been used in many initial procedures, such that tulip couplers are already present in many patients. In accordance with many competing systems, such tulip couplers must be removed in order to perform a revision surgery. In accordance with other competing systems, a large amount of additional bulky hardware must be implanted to build off of existing tulip couplers. In this regard, the additional bulky hardware and new anchor insertions can cause significant damage to a patient’s spine and can extend surgery duration. In contrast, in accordance with some embodiments of the present systems and methods, one or more of the tulip couplers can remain in place, a collet 130 can be attached to the connector (as described above), and an additional portion (e.g., a second portion 100b, a third portion 100c, or any other portion) of the coupling assembly 100 can be added directly onto the end of the connector, removing the need for bulky equipment or for removal of the tulip couplers. By way of non-limiting illustration, FIG. 2A shows a coupling assembly 100 including a first portion 100a having a tulip coupler 152 joined to a second portion 100b having a tulip coupler 152 by a connector 120 in the form of a rod, as may have been used in an earlier procedure to fuse a first vertebra to a second vertebra. FIG. 2B shows a collet 130 disposed on a first end 122 of the connector 120. FIG. 2C shows a press-on coupler 162 added to the coupling assembly 100, joining the second portion 100b (and by extension, the first portion 100a) to a third portion 100c (having a third anchor 102c) to reach an additional level of the patient’s spine.

According to some embodiments, the coupler 150 (e.g., the press-on coupler 162 that can be pressed or snapped onto anchor head 108 or other component) is configured to be installed while other components of the coupling assembly 100 are already in place. For example, in some cases, the coupler is configured to be coupled to an anchor 102 while the anchor is already anchored in the patient’s spine. In some cases, the coupler is configured to be coupled to a connector 120 that is already joining other sections of the coupling assembly together. By way of non-limiting illustration, FIG. 5A shows some embodiments in which a first portion 100a (having a first anchor 102a) of a coupling assembly 100 is connected to a second portion 100b (having a second anchor 102b) of the coupling assembly via a connector 120. A third anchor 102c (as shown) can be installed in an adjacent vertebra. FIG. 5B shows that, in accordance with some embodiments, a collet 130 is installed on the connector 120, and then a coupler 150 (in the form of a press-on coupler 162 or any other suitable coupler) can be coupled to the collet (and correspondingly, the connector) and to the new anchor 102 without removing either. In some embodiments, this results in a unified, three-level coupling assembly 100, as shown in FIG. 5C.

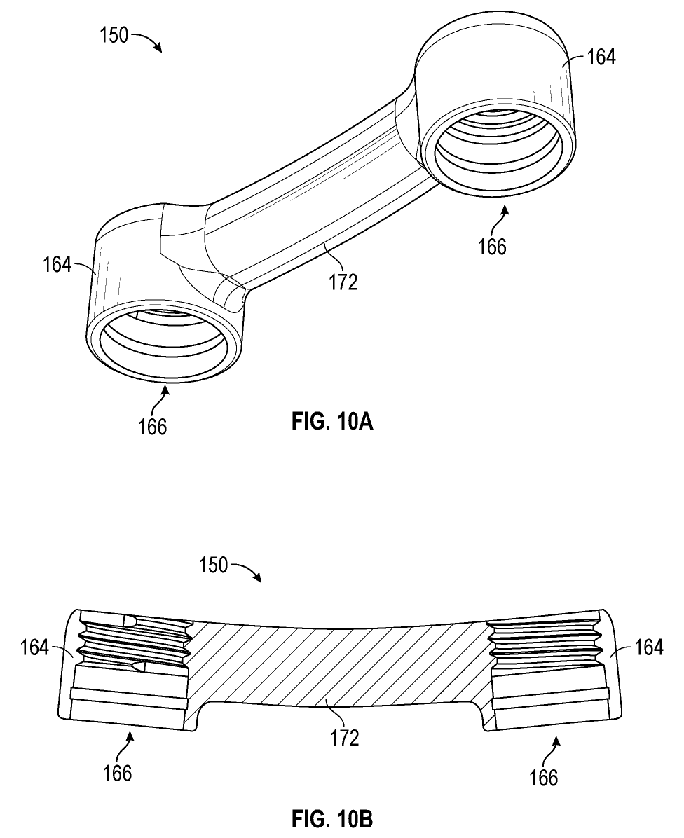

In line with the foregoing, some embodiments of the coupler 150 include one or more anchor receptacles 164 configured to receive an anchor 102. The anchor receptacle can be configured to receive the anchor in any suitable manner. For example, some embodiments are configured to receive the anchor through the top (e.g., shaft-first), such that the shaft extends out from the bottom of the anchor receptacle (like some embodiments of the tulip 156 of a tulip coupler 152 as described above). However, in some embodiments, a bottom of the anchor receptacle includes one or more openings 166 configured to receive the anchor head-first and correspondingly retain the anchor, such that the coupler can be “snapped on” to the anchor or otherwise coupled to the anchor (e.g., coupled via an interference fit, a snap fit, a friction fit, a press fit, a detent fit, a resilient fit, an elastic fit, a compliant fit, a keyed fit, a dovetail fit, or any other type of retaining fit or locking fit for coupling the anchor to the anchor receptacle). By way of non-limiting illustration, FIGS. 7C-7D show some embodiments of an anchor 102 with its head 108 pressed into and coupled to an anchor receptacle 164. By way of additional illustration, FIGS. 10A-10B show some examples of couplers 150 having anchor receptacles 164 having openings 166 formed in the bottoms thereof (such that the anchor receptacles can receive a head 108 of an anchor 102).

In some embodiments, the anchor receptacle 164 is configured to receive the anchor 102 polyaxially. For example, in some cases, the anchor receptacle can receive the anchor with a long axis of the anchor and a long axis of the anchor receptacle being parallel to each other, or being at an angle with respect to each other, such as between 0° and 45° (or any subrange thereof, such as between 0° and 15°, 0° and 20°, 0° and 25°, 0° and 30°, or any other suitable subrange).

According to some embodiments, the anchor receptacle 164 includes one or more additional features for forming a strong coupling between the anchor head 108 and the receptacle. For example, some embodiments include one or more interfaces, locks, rims, grooves, knurled surfaces, protuberances, recesses, notches, contours, or other features configured to provide a strong coupling. For example, some embodiments of the anchor receptacle include one or more retention grooves 168.

Where the anchor receptacle 164 includes a retention groove 168, the retention groove can have any suitable configuration for improving the strength of a coupling between the anchor 102 (e.g., the anchor head 108) and the anchor receptacle. In some cases, the retention groove is configured to allow the coupling to maintain its polyaxiality. For example, in some cases, the anchor head is configured to be inserted through the opening 166 in the bottom of the anchor receptacle at a variety of different angles (as discussed above), and then to be maintained at the insertion angle. In some embodiments, one or more portions of the anchor head (e.g., a rounded portion, a portion where the diameter is at a maximum, or any other suitable portion) is configured to be at least partially disposed within the retention groove. In some cases, this creates a local potential energy minimum, thereby requiring the potential energy to increase—which requires a greater application of force—before the anchor head can separate from (or move with respect to) the anchor receptacle. By way of non-limiting illustration, FIG. 9C shows a coupling assembly 100 having a number of anchor receptacles 164, wherein the anchor receptacle includes one or more retention grooves 168 formed on an inner surface thereof, and wherein some of the anchor receptacles are coupled to anchors 102 having anchor heads 108 inserted into the anchor receptacles such that the anchor heads bulge or extend into the corresponding retention grooves to form a tight coupling (even where the anchors are disposed at different angles).

Where a retention groove 168 is included, the retention groove can have any suitable size, position, orientation, depth, shape, or other characteristics that enable it to strengthen the coupling between the anchor head 108 and the anchor receptacle 164.

The retention groove 168 can have any suitable shape. For example, in some embodiments, the retention groove extends all the way around an inner perimeter of the anchor receptacle, thereby forming an unbroken groove in an annular shape (or in a shape that otherwise follows the inner perimeter of the anchor receptacle), and thereby being configured to receive an outer perimeter of a portion of the anchor head. That said, in some cases, the retention groove only extends part way around (e.g., forming a partial ring, a broken ring, perforations, or another shape or shapes configured to receive part of the anchor head, such as a portion of an outer perimeter of the anchor head) when the anchor head is forced into the anchor receptacle. By way of non-limiting illustration, FIGS. 7A-7B each show part of a retention groove 168 that, if visible, would extend all the way around the inner perimeter of the associated anchor receptacle 164. Additionally, a cross-sectional shape of the retention groove can be any suitable shape, such as square, squared, rectangular, semi-circular, triangular, polygonal, tiered, C-shaped, or any other regular or irregular shape. In some cases, the shape of the groove is configured to correspond with a curvature of the anchor head 108. In some cases, the shape of the groove does not correspond with a curvature of the anchor head, such that an uneven pressure is applied to the anchor head (e.g., one or more edges of the groove press against or “pinch” the anchor head or a portion of the anchor head bulges into the groove, both of which in some cases help to prevent slippage or other unintentional adjustment of the anchor’s angle with respect to the anchor receptacle). By way of non-limiting illustration, the retention grooves 168 shown in FIGS. 7A-7B have a generally square or squared cross-sectional shape.

The retention groove 168 can have any suitable orientation. According to some embodiments, the retention groove is approximately parallel to the opening 166 in the bottom of the anchor receptacle 164. That said, in some embodiments, the retention groove is set at an angle, such as between 1 and 35 degrees offset from parallel to the opening. In some embodiments, the retention groove undulates, is stepped, is angled, zig-zags, or otherwise extends around a portion of the anchor receptacle.

The retention groove 168 can have any suitable size. For example, some embodiments of the retention groove have a depth (e.g., extending a depth into the sidewall of the anchor receptacle 164) of between 10 µm and 2 mm, or within any subrange thereof (e.g., between 50 µm and 1.5 mm, between 100 µm and 1.2 mm, between 250 µm and 1 mm, or any of suitable subrange). In some embodiments, the retention groove has a width (e.g., a distance from a top of the retention groove to a bottom of the retention groove) of between 0.1 mm and 3 mm, or any subrange thereof (e.g., 0.25 mm - 2.5 mm, 0.5 mm – 2 mm, 1 mm ± 0.5 mm, or any other suitable subrange).

The retention groove 168 can also have any suitable position. For example, in some embodiments, the retention groove is positioned between 0.2 mm and 5 mm above the bottom (e.g., away from a lower edge) of the anchor receptacle 164, or any subrange thereof (e.g., between 0.3 mm and 1 mm, 0.5 mm and 1.5 mm, 0.75 mm and 2.5 mm, or any other suitable subrange). Indeed, some embodiments of the retention groove are positioned in a lower third of the anchor receptacle. In some embodiments, the retention groove is positioned about halfway between the bottom of the anchor receptacle and a threading 170 (as discussed in more detail below). In some embodiments, the retention groove is roughly even with a lower edge of a connector portion 172 or a locking element 178 (in each case, as discussed in additional detail below). In some cases, the positioning of the retention groove helps achieve a strong coupling while providing appropriate angles for the anchor 102 and connector portion, and taking into account the forces experienced by the coupling assembly 100 in physiological conditions. In some cases, the positioning of the retention grooves helps ensure that the anchor head 108 is appropriately seated within the anchor receptacle and is able to achieve the desired range of polyaxial connection (as discussed above) prior to being selectively locked in position. By way of non-limiting illustration, FIGS. 7A-7D show the positioning of retention grooves 168 in accordance with some embodiments (e.g., roughly even with a lower edge of a connector portion 172 or a locking element 178, approximately in a lower third of the anchor receptacle 164, approximately halfway between the bottom of the anchor receptacle 164 and a threading 170, and otherwise positioned to provide a strong coupling with correct angles for the anchor 102 and the connector portion 172).

According to some embodiments, the retention groove 168 is positioned such that a substantial portion (e.g., at least 50%, 60%, 70%, or 75%) of the anchor head 108 is fully disposed within the anchor receptacle 164 when an outer surface of the anchor head is disposed within the retention groove. For example, in some cases, the anchor head has one or more circumferences where the diameter is greatest (e.g., due to the curved surface of some embodiments of the anchor head), and in some cases, such circumferences become disposed within the retention groove when the anchor head is inserted into the anchor receptacle. Thus, in some cases, the retention groove is positioned such that when the anchor head is retained in such manner, the anchor has an appropriate height relative to the coupler 150 (e.g., to the connector portion 172).

According to some embodiments, the anchor receptacle 164 includes a threading 170 or any other suitable type of interface. For example, some embodiments of the anchor receptacle are configured to receive a set screw 160 (or another suitable component) in a top thereof. For example, FIGS. 7A-7D show anchor receptacles 164 with threading 170 configured to receive a set screw, a tool, or another component, in accordance with some embodiments.

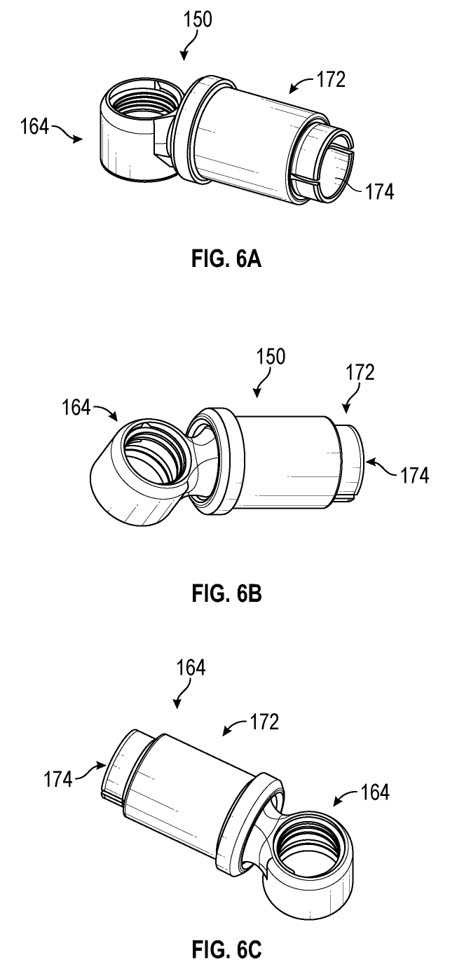

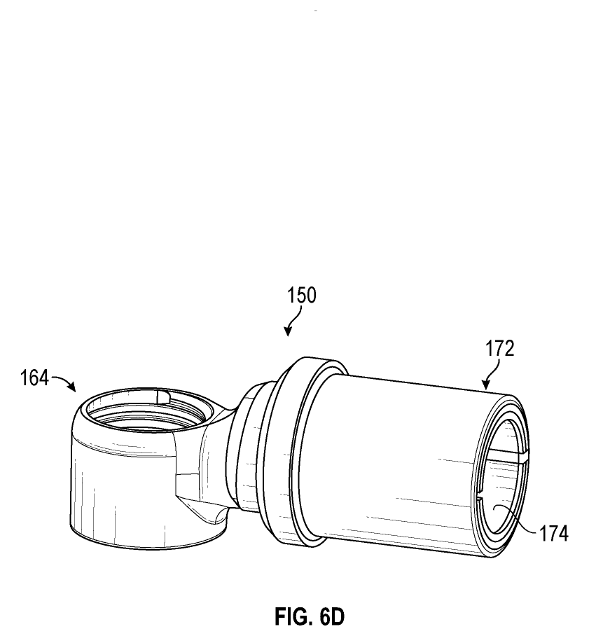

According to some embodiments, the coupler 150 (e.g., the press-on coupler 162) includes one or more connector portions 172. The connector portion can include any component or feature of the connector 120 as discussed above. Indeed, in some embodiments, the connector portion includes or functions as a connector (e.g., spanning a distance between two or more portions of the coupling assembly 100), so any of the disclosure above relating to a connector can apply to a connector portion, and any of the disclosure here relating to a connector portion can also be applied to a connector. That said, in some embodiments, the connector portion is coupled to (e.g., is integrally formed with, selectively couplable to, part of, included with, or otherwise coupled to) the anchor receptacle 164 to form the coupler (or to form a main body or other portion thereof). By way of non-limiting illustration, FIGS. 6A-6D show some embodiments of couplers 150 with anchor receptacles 164 and connector portions 172 integrally formed together into one main body.

According to some embodiments, the connector portion 172 is configured to couple to another connector 120. While the connector portion can couple to another connector in any suitable manner (e.g., via one or more nails, screws, threaded engagements, bolts, staples, eyelets, magnets, hook-and-loop fasteners, adhesives, welds, interference fits, friction fits, mechanical engagements, tongue-and-groove connections, snaps, buttons, zippers, ties, rivets, stakes, or any other coupling mechanism), some embodiments include one or more connector receptacles 174 configured to receive a portion (e.g., an end) of the other connector.

Where the connector portion 172 includes a connector receptacle 174, the connector receptacle can include any suitable component for receiving an end of the connector 120 (e.g., as coupled to a collet 130) and for coupling the connector portion to the connector. Accordingly, some embodiments include any suitable feature analogous to any feature of the anchor receptacle 164. For example, some embodiments include one or more openings 166, retention grooves 168, threadings 170, measurements, polyaxial coupling capabilities, or other components or features as disclosed above in connection with the anchor receptacle. By way of non-limiting illustration, FIG. 1F shows, in accordance with some embodiments, a connector portion 172 with a connector receptacle 174 with an opening 166 configured to receive a first end 122 of a connector 120 having a collet 130 coupled thereto (thereby able to selectively couple polyaxially), the connector receptacle further comprising a threading 170 configured to receive a set screw 160 to retain the connector 120 in place with respect to the connector portion 172.

The connector receptacle 174 can be configured to receive (and couple to) the connector 120 (e.g., with the collet 130) in any suitable manner. For example, some embodiments have a first diameter of a sufficient size to allow the collet to freely enter the connector receptacle, and some embodiments are configured to switch from a first configuration with the first diameter to a second configuration with a second diameter, wherein the second diameter is smaller than a diameter of the collet, such that the collet becomes compressed or otherwise lodged in the connector receptacle, thereby creating a coupling. In this regard, some embodiments of the connector receptacle include one or more receptacle compression features 176 to allow for compression from the first diameter to the second diameter. Where one or more receptacle compression features are included, the receptacle compression features can include any slot 156, gap 136a, slit 136b, thinned wall, compression feature 136, or any other component or feature of any other component that allows the connector receptacles to decrease in diameter. By way of non-limiting illustration, FIGS. 7A-7B show some embodiments of connector receptacles 174 having gaps 176 configured to compress to shift the connector receptacles from a first configuration to a second configuration with a smaller diameter.

According to some embodiments, the coupler 150 includes one or more locking elements 178. The locking element can include any suitable component configured to facilitate the compression of the connector receptacle or to otherwise lock in place the coupling between the connector portion 172 and the connector 120. For example, some embodiments of the locking element include one or more: rings, tubes, clamps, clasps, crimps, sleeves, pins, catches, textured surfaces, or other components configured to facilitate the coupling.

According to some embodiments, the locking element 178 is configured to selectively slide from a first position (in which the connector receptacle 174 is not coupled to the collet 130) to a second position (in which the connector receptacle is forced to compress around the collet to form a coupling).

According to some embodiments, the locking element 178 includes one or more tubes, rings, sleeves, or other annular components configured to surround (partly or completely) at least a portion of the connector portion 172.

The locking element 178 can have any suitable dimensions. For example, in some cases, it has an inner diameter of a size configured to hug or compress all or a portion (e.g., the connector receptacle 174) of the connector portion 172. In some cases, the connector receptacle has a slightly larger diameter than another part of the connector portion, such that when the locking element slides over the connector receptacle, the connector receptacle compresses. In some cases, the locking element compresses the connector portion along any length over which the locking element is disposed, such that when the locking element is disposed over the connector receptacle, this results in compression and (if the collet 130 is in place) a resulting coupling. In some embodiments, the locking element has a variable diameter (e.g., inner diameter, outer diameter, or both). For example, in some cases, an inner diameter near the connector receptacle is smaller than an inner diameter farther from the connector receptacle to provide for greater compression at the connector receptacle when the locking element is in the locking position. In some embodiments, the outside diameter is variable, which in some cases includes the formation of one or more additional features (such as a flange 180, as discussed in more detail below).

The locking element 178 can also have any suitable length. For example, the locking element can have a length of between 1 mm and 10 mm, or any subrange thereof (e.g., between 1 mm and 5 mm, 1.5 mm and 3.5 mm, 1 mm and 2 mm, or any other suitable subrange). In some cases, the locking element has a length near a length of the connector portion 174 (e.g., between 50% and 95% of the length of the connector portion, or any subrange thereof). In some cases, the locking ring has a relatively small length compared to the connector portion (e.g., between 5% and 50% of the length of the connector portion, or any subrange thereof). By way of non-limiting illustration, FIGS. 7A-7D show some embodiments of locking elements 178 having lengths that are greater than 70% of the lengths of the corresponding connector portions 172. By way of further non-limiting illustration, FIGS. 8A-8C show some embodiments of locking elements 178 having lengths that are less than 40% of the lengths of the corresponding connector portions 172.

According to some embodiments, the locking element 178 includes one or more flanges 180. In this regard, the flange can include any suitable portion of increased thickness or other element configured to increase the compression strength, durability, or profile (e.g., for better interaction with a tool) of the locking element. By way of non-limiting illustration, FIGS. 7A-7D show some embodiments of locking elements 178 having flanges 180.

According to some embodiments, a locking element 178 is provided in the form of a set screw 160. Indeed, in some cases, the set screw is configured to be screwed into a threaded portion of the coupler 150 such that it presses into the collet 130, thereby retaining the collet within the connector receptacle 174. By way of non-limiting illustration, FIG. 1D shows a coupler 150 having a connector receptacle 174 receiving an end of a connector 120 with a collet 130 coupled thereto, wherein a locking element 178 in the form of a set screw 160 is used to secure the collet in place, thereby coupling the coupler 150 to the connector 120.

Although in some embodiments, the connector receptacle 174 is fluidically connected with the anchor receptacle 164, in some embodiments, they are not fluidically connected. For example, in some cases, the connector portion 172 is substantially solid (or otherwise does not include a lumen passing therethrough).

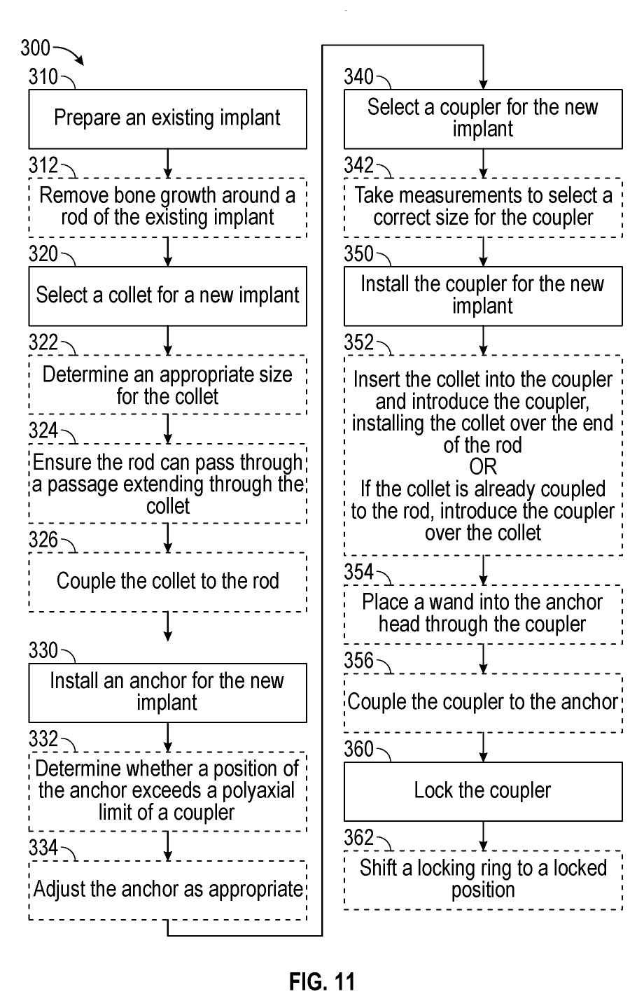

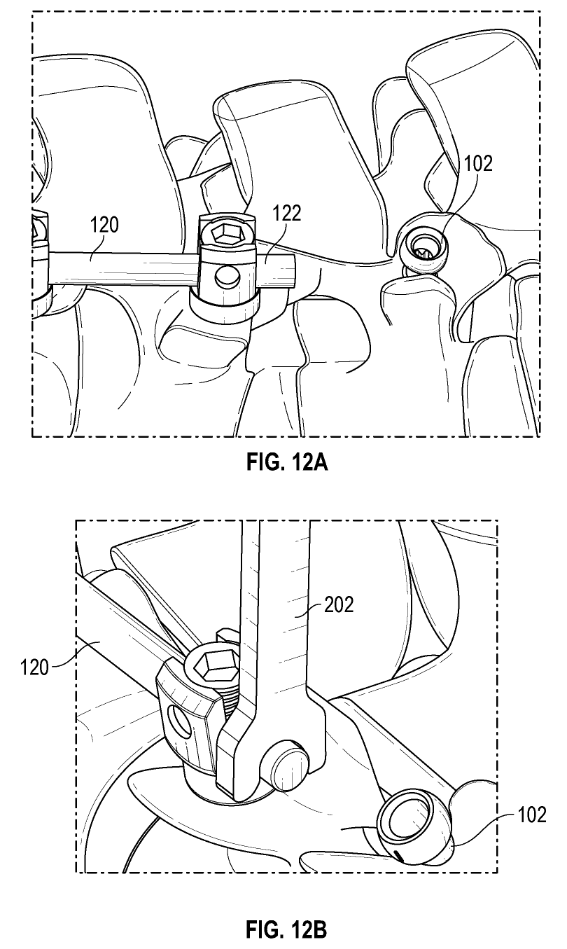

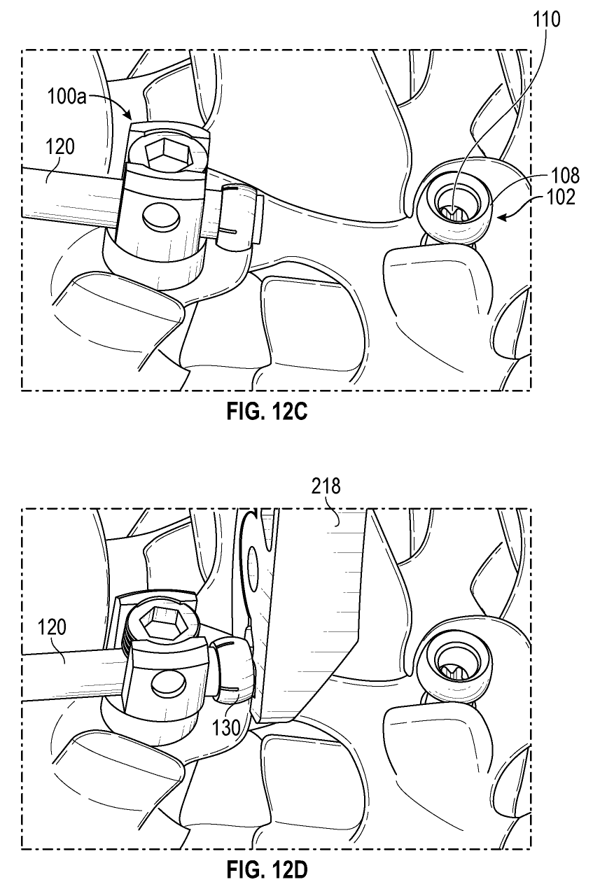

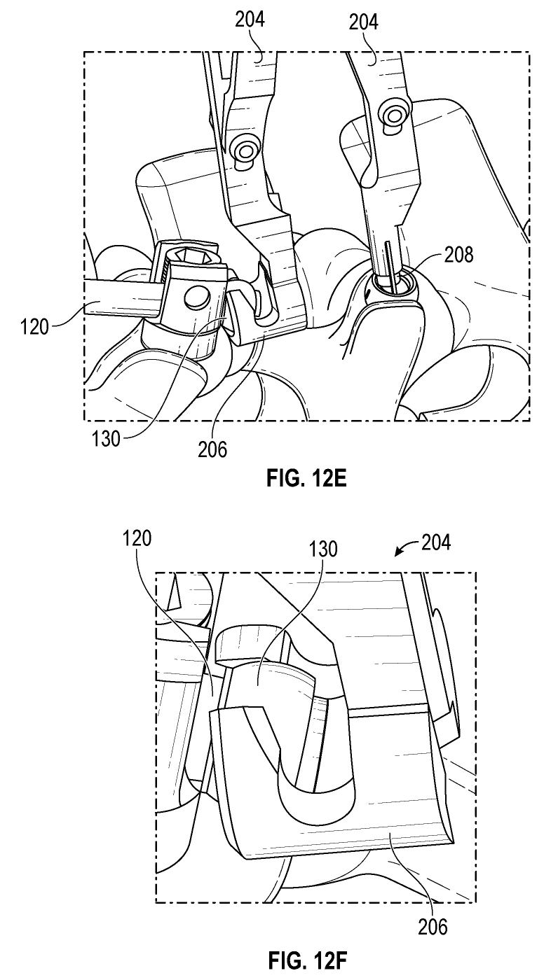

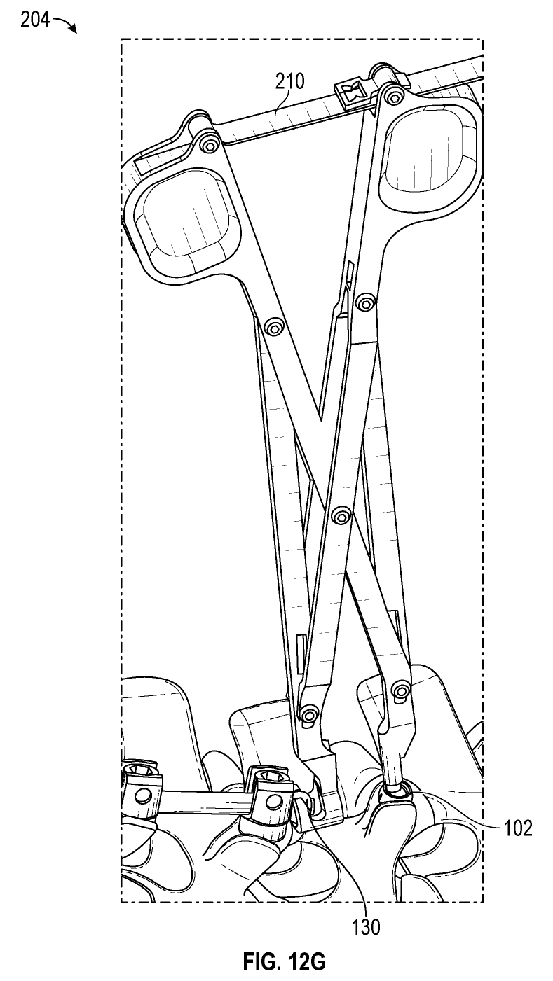

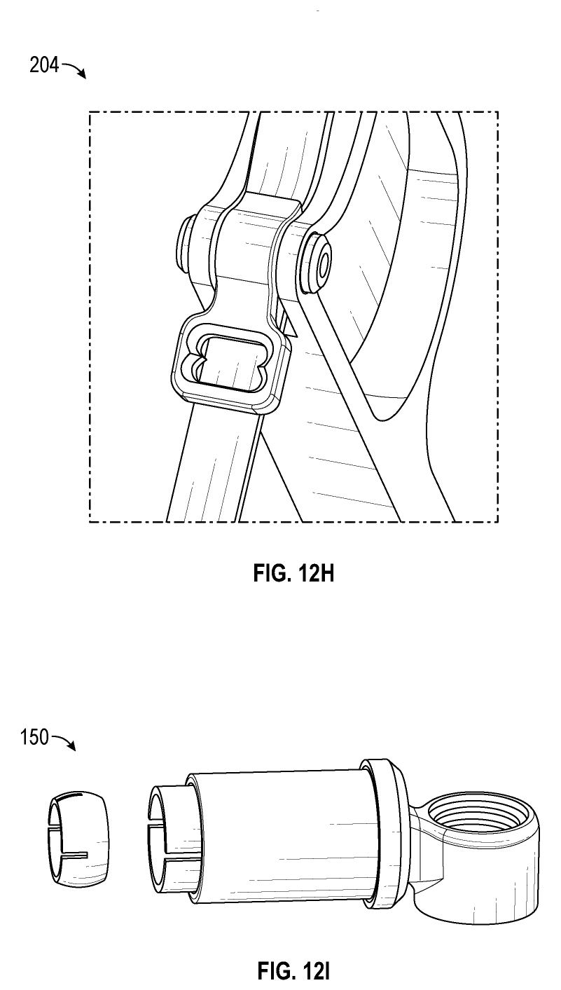

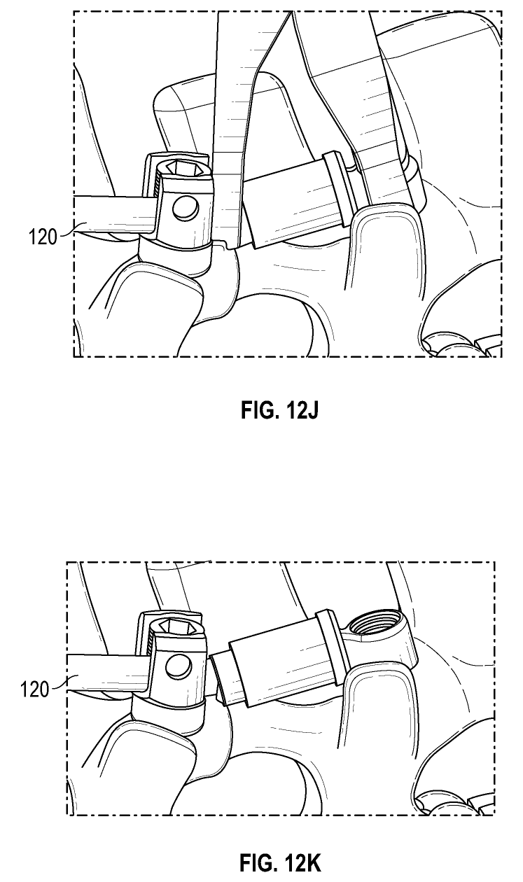

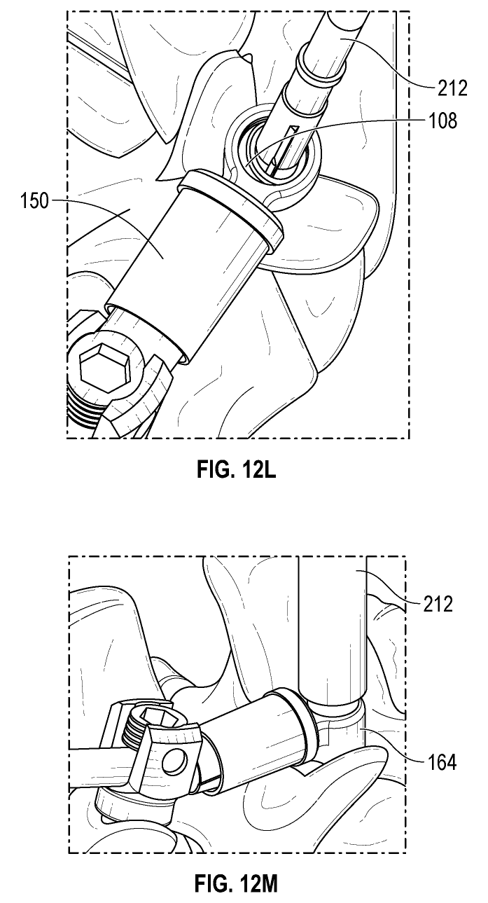

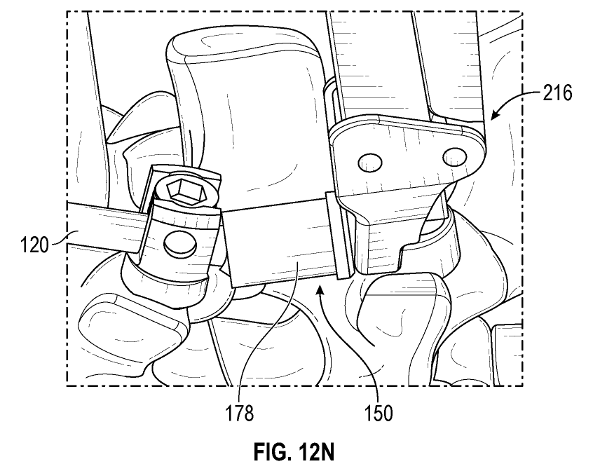

In accordance with some embodiments, one or more methods of providing or removing a coupling assembly 100 are provided. For example, some embodiments include one or more methods of providing a coupling assembly.

Where methods for providing a coupling assembly is provided, the methods can include any suitable action for making use of any of the components discussed above. Indeed, any and all of the components in the figures, embodiments, implementations, instances, cases, methods, applications, iterations, and other parts of this disclosure can be combined in any suitable manner. Moreover, the various portions of the described methods can be reordered, omitted, substituted, repeated, performed in parallel, performed in series, performed so as to be overlapping in time, modified, or otherwise changed in any suitable manner. That said, a non-limiting example of some embodiments of a method 300 for providing a coupling assembly is shown in FIG. 11, with various portions of the method being illustrated in FIGS. 12A-12N.

In some embodiments, the method 300 includes performing an initial procedure to insert a first implant. That said, in some cases, the method includes performing a revision procedure to add an additional portion of an implant to an existing implant.

As shown by box 310 (of FIG. 11), some embodiments include preparing an existing implant. This can include any suitable preparation, such as cleaning, disinfecting, adjusting a position of, or otherwise ensuring that the existing implant is ready for extension (e.g., for an additional implant or implant portion to be added on to the existing implant). In some embodiments (as shown in box 312 of FIG. 11), the method includes removing bone growth (or other physiological components or buildup resulting from the implant’s time inside the patient) from around a rod or another connector 120 of the existing implant. In some embodiments, a minimum amount of overhang of the existing connector is prepared (such as at least 6 mm of connector or any other suitable amount). In some cases, this includes selecting an appropriate extension (e.g., a collet 130 having an extension component) to ensure that the appropriate amount of overhang can be achieved. In some cases, the overhang is between 1 mm and 1.5 cm (and any subrange thereof, such as between 5-10 mm, 6-8 mm, or another suitable amount). By way of non-limiting illustration, FIG. 12A shows an example of a connector 120 with a first end 122 that has been prepared.

According to some embodiments, the method 300 includes selecting a collet for the new implant (as shown by box 320 of FIG. 11). In some cases, this includes determining an appropriate size for the collet (as shown by box 322 of FIG. 11). While this can be done in any suitable manner (e.g., using any suitable tool for measurement, via trial and error, or otherwise), some embodiments include using a set of rod trials 202 or another suitable tool (e.g., by finding the smallest size trial that fits over the rod). In some cases, a collet size is selected based on the trial measurements. By way of non-limiting illustration, FIG. 12B shows a determination of the size of a connector 120 using a rod trial 202.

As shown by box 324 (of FIG. 11), some embodiments include ensuring that the connector 120 can pass through a passage extending through the collet 130 (or, in other words, that the collet is appropriately sized to slide onto the rod).

As shown by box 326 (of FIG. 11), some embodiments include coupling the collet 130 to the connector 120. This can be done in any suitable manner, such as using any coupling mechanism as discussed above. For example, some embodiments of the collet are crimped, welded, soldered, adhered, pinned, screwed, frictionally fit, or otherwise coupled to the connector. In some cases, this coupling is performed prior to attachment of a coupler 150, and in some cases, the coupling is performed concurrently with attachment of the coupler (e.g., in some cases, sliding the locking element 178 not only couples the connector receptacle 174 to the collet, but it also compresses the collet, causing the collet to couple to the connector).

In some embodiments, coupling the collet 130 to the connector 120 includes deforming the collet or the connector (or both), or otherwise shifting the collet or the connector from a decoupled position to a coupled position. This can be done in accordance with any of the functionality of connectors or collets as described above. That said, in some embodiments (e.g., as shown in FIG. 12D), a crimper 218 is used to crimp or otherwise deform an end of a connector 120 or a collet 130 to ensure that the collet is firmly coupled to the connector.

As shown by box 330 (of FIG. 11), some embodiments of the method 300 include installing an anchor 102 for the new implant portion. As discussed above, any suitable anchor can be used, and the anchor can be installed in any suitable manner. That said, as shown by boxes 332 and 334 (of FIG. 11), some embodiments include determining whether a position of the anchor exceeds a polyaxial limit of the coupler 150, and adjusting the anchor as appropriate. While this can be done in any suitable manner, some embodiments include using extension calipers, such as by placing a foot 206 of the extension calipers over the end of the connector 120 and a tip 208 of the extension calipers into a recess 118 of an anchor 102 (e.g., to couple the tip to a drive 110 of the anchor). In some cases, if the extension calipers cannot pivot sufficiently to connect to the connector and anchor, this indicates that the current anchor position exceeds the polyaxial limit of the coupler. The height or other position of the anchor can then be adjusted until the extension calipers connect properly. For example, FIG. 12C shows an example of an anchor 102 installed in an approximately appropriate position with respect to a first portion 100a of a coupling assembly, and FIG. 12E shows extension calipers 204 with a foot 206 coupled to the end of a connector 120 (and collet 130) and a tip 208 coupled to the anchor 102. FIG. 12F shows a closeup of a foot 206 of extension calipers 204 as coupled to a collet 130 on a connector 120.

According to some embodiments, as shown in box 340 (of FIG. 11), the method 300 includes selecting a coupler 150 for the new implant (or the new, or revision, portion of the implant). Indeed, some embodiments (as shown by box 342 of FIG. 11) include taking measurements in order to ensure that a coupler of the correct size (e.g., having a connector portion 172 of the desired length) can be selected. Although the measurements may be taken in any suitable manner, some embodiments of the extension calipers 204 include one or more scales 210 configured to measure and convey a measurement of the distance between the anchor 102 and the collet 130. By way of non-limiting illustration, FIG. 12G shows extension calipers 204 having a scale 210 configured to measure a distance between an anchor 102 and a collet 130 so that an appropriately sized connector (e.g., a connector 120 as shown in FIG. 12I) can be selected.

As shown by box 350 (of FIG. 11), some embodiments of the method 300 include installing the coupler 150 for the new portion of the implant. This can be done in any suitable manner, as the skilled artisan will recognize based on the descriptions of the collet 130 and coupler 150 as provided above. For example, as shown in box 352, some embodiments include inserting the collet into the coupler, and then subsequently introducing the coupler and inserting the collet (which is, in such cases, already in the coupler) over the end of the connector 120. In some cases, a tool (such as an extension assembler) is used to press the collet into the selected coupler. Additionally, although a collet can be inserted into the coupler in any suitable manner, in some cases where a collet with slots on one side is used, the slotted side faces away from the coupler when inserted (e.g., as shown in FIG. 12I) to allow for the collet to engage the connector only as desired, when the locking element 178 is shifted to the locking position (e.g., in some cases, the slots facing away from the coupler can help prevent premature engagement).

As further shown by box 352 (of FIG. 11), in some cases (e.g., where the collet 130 is already coupled to the connector 120), installing the coupler 150 includes introducing the coupler over the collet.

As shown by box 354 (of FIG. 11), some embodiments of the method 300 include placing a wand 212 into the anchor head 108 (e.g., into a recess 118 or drive 110 of the anchor head). In some cases, the wand is then used to guide the coupler 150 to its proper location. In some cases, the wand is placed through the coupler after the coupler is already in place (or approximately in place). In some embodiments, the wand is used to guide another tool 200, such as an anchor locker 214, to a location where it can be used to lock the coupler to the anchor 102. For example, in some cases (e.g., as shown in FIG. 12L), the wand 212 is inserted into the anchor head (before or after the coupler 150) is in place, and then (e.g., as shown in FIG. 12M) the hollow anchor locker 214 is slid over the wand and used to press the anchor receptacle 164 onto the anchor to form a coupling.

As shown by box 360 (of FIG. 11), some embodiments of the method 300 include locking the coupler 150, such as by placing the locking element 178 into the locked position (as shown by box 362). Although this can be done in any suitable manner, in some cases this is done using a tool 200, such as a connector locker 216. For example, FIG. 12N shows a connector locker 216 being used to shift a locking element 178 to a locked position, thereby locking a coupler 150 in place with respect to a connector 120.

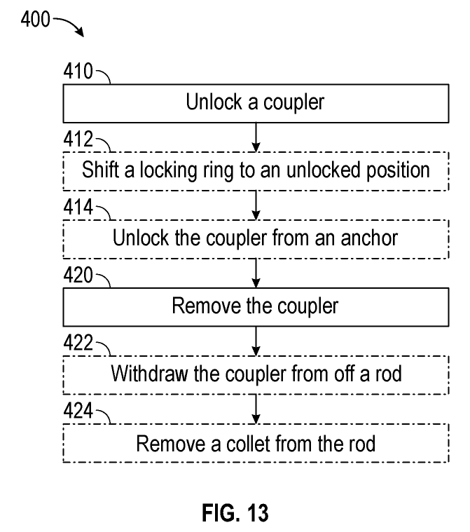

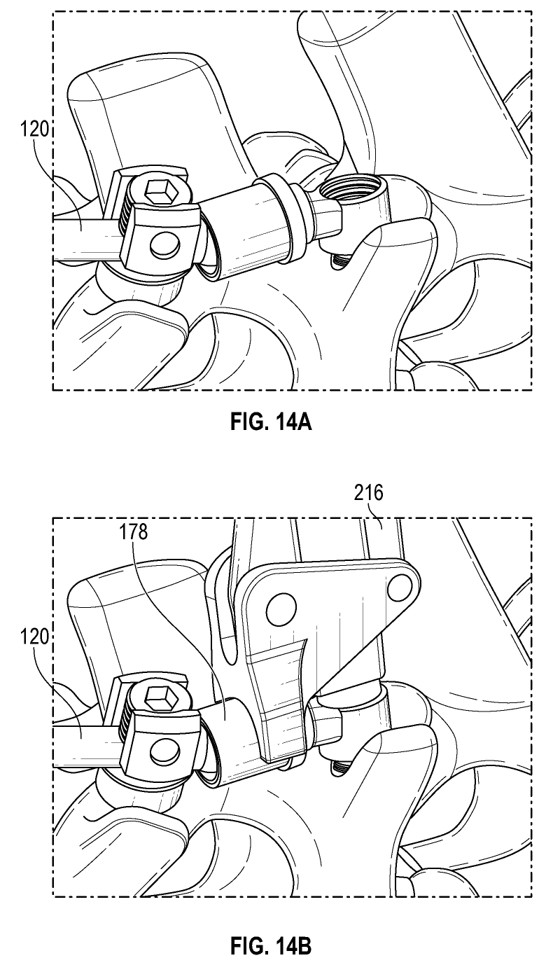

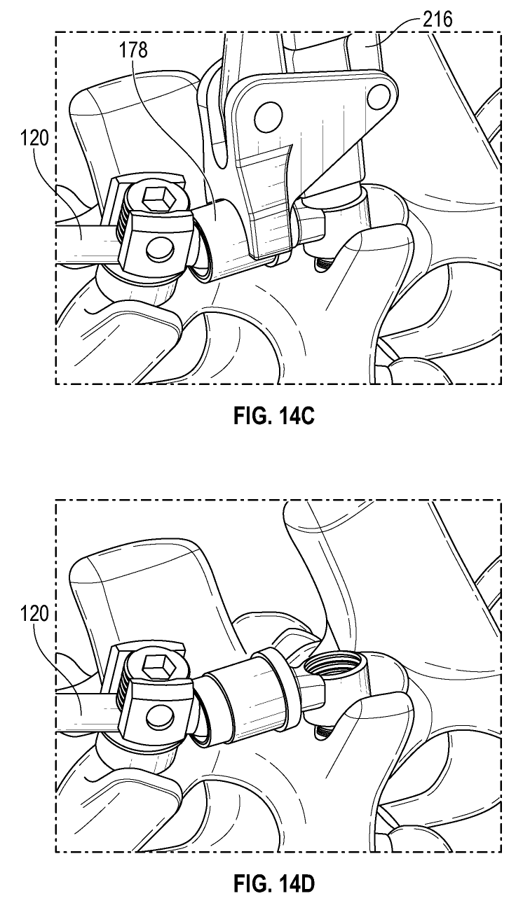

In accordance with some embodiments, one or more methods 400 for removing a coupling assembly 100 (or a portion thereof) is provided. In this regard, a reverse analog of any of the portions of the method 300 for providing a coupling assembly can be implemented, or the coupling assembly can otherwise be removed. By way of non-limiting illustration, FIG. 13 describes some embodiments of a method 400 including unlocking a coupler 150 (as shown by box 410), which may further include shifting a locking element 178 to an unlocked position (as shown by box 412) or unlocking the coupler from an anchor 102 (as shown by box 414), and removing the coupler (as shown by box 420), which may further include withdrawing the coupler from off a connector 120 (as shown by box 422) or removing a collet 130 from the connector (as shown by box 424). In this regard, one or more tools 200, in accordance with some embodiments, are configured to operate to selectively disassemble the coupling assembly. For example, in some cases (as shown by FIGS. 14B-14C), a connector locker 216 is configured to shift a locking element 178 to an unlocked position.

In some embodiments, one or more kits are provided, with the kit including any of the components discussed herein, including multiple of a certain type of component with various sizes or configurations. For example, some embodiments of the kit include one or more: anchors 102 (in some cases, with varying: sizes; shafts 104; threadings 106; heads 108 (e.g., with different shapes, drives 110, or otherwise; retention features 112; tips 114; channels 116; recesses 118; or other configurations, as discussed above); connectors 120 (in some cases, with varying: sizes; shapes; ends (e.g., a first end 122 and a second end bwith any suitable configuration); diameters 126 (whether uniform or variable); lengths 128; retention features 140; deformable portions 142; rims 144; expanders 146; caps 148; or other varying features); collets (e.g., with varying: sizes, compression features 136; retention features 140; deformable portions 142; rims 144; expanders 146; caps 148; or other features); couplers 150 (including tulip couplers 152 or press-on couplers 162 (or both)), having any suitable features, such as: retention grooves 168; threading 170; connector portions 172; connector receptacles 174; receptacle compression features 176; locking elements 178; flanges 180; sizes; shapes; and other configurations; tools 200 (e.g., rod trials 202; extension calipers 204; wands 212; anchor lockers 214; connector lockers 216; crimpers 218; and any other suitable tools. The kit can include any other suitable components as well, in any suitable number and configuration, for giving practitioners a convenient experience in providing or removing an implant assembly 100.

The implant assembly 100, methods 300, 400, or kit can be modified in any suitable manner. For example, in some embodiments, one or more of the components is custom-formed to a specific shape or configuration to adapt to a unique curvature or other attribute of the patient’s spine.

As another example of a suitable modification, in some embodiments, the anchors 102 comprise one or more hooks (or any other suitable anchor) instead of or in addition to one or more bone screws. Thus, in some embodiments, the described implant assembly 100 is configured to attach to adjacent spinal levels through the use of one or more hooks instead of, or in addition to, one or more bone screws.

As another example, some embodiments of the coupling assembly 100 are configured to use only tulip couplers 152 (e.g., without press-on couplers 162), or are otherwise configured for expanded use of one or more tulip couplers. To illustrate, some embodiments utilize one or more couplers 150 that lack an anchor receptacle 164, but which have a connector portion 172 configured to be used in connection with a tulip coupler. In some such cases, a collet 130 can be placed on the end of a connector 120, and then a coupler (with or without an anchor receptacle) can be coupled thereto as discussed herein (in some cases, using a locking element 178, such as a locking ring, a set screw 160, or another coupling mechanism). Subsequently, the connector portion of said coupler can be disposed within an additional coupler (e.g., a tulip coupler), thereby joining the end of the connector to the tulip coupler using the coupler without an anchor receptacle as an intermediate component or adapter. By way of non-limiting illustration, FIG. 1G shows some embodiments of a coupling assembly 100 having a connector 120 in the form of a rod, a collet being disposed on the end thereof (not shown, but illustrated in FIG. 1F as collet 130). The collet and the end of the rod in this figure are then disposed within a connector receptacle 174 of a first coupler 150a (the first coupler not having an anchor receptacle, in this case). A locking element 178 in the form of a set screw 160 is used to retain the collet within the coupler (the set screw pushes against the collet, creating friction and a strong connection, as shown in FIG. 1F). The first coupler includes a connector portion 172 (in this case, integrally formed with the first coupler, but which could also be selectively couplable to the first coupler), which functions as a rod (in this case, having a similar diameter to the connector 120). The connector portion 172 in FIG. 1G is then disposed within a slot 158 of a second coupler 150b in the form of a tulip coupler 152. A set screw 160 retains the connector portion 172 within the slot 158.

As illustrated by the example in the previous paragraph, many different types of couplers 150 can be used in various embodiments. Some couplers include no anchor receptacles 164 (such as the first coupler 150a in FIG. 1G or the coupler 150 of FIG. 1E), and some couplers include one anchor receptacle 164 (such as the tulip coupler 152 or the press-on coupler 162 of FIG. 1A), and some couplers include more than one anchor receptacle 164 (such as the couplers 150 of FIGS. 10A-10B, which each contain two anchor receptacles 164). Similarly, some couplers include no connector receptacles 174 (such as the couplers 150 of FIGS. 10A-10B, which contain no connector receptacles, but which contain connector portions 172 that could be disposed within a slot 158 of a tulip coupler 152), some couplers include one connector receptacle 174 (such as the couplers 150 shown in FIGS. 6A-6D), and some couplers include more than one connector receptacle 174 (such as the second coupler 100b shown in FIG. 8A, which includes two connector receptacles 174). Thus, some embodiments of the present systems and methods allow for attachment of connectors 120 or connector portions 172 to other connectors or connector portions, with placement of anchor receptacles 164 as needed to align with anchors 102.

The coupling assembly 100 can include any other suitable feature. Some embodiments of the coupling assembly provide a great amount of flexibility in terms of installation and removal in both initial procedures and follow-up procedures, thereby providing a number of advantages over some competing devices. In this regard, some competing devices are much more limited in terms of performance of revision procedures. For example, when many competing implants fail, or when one or more adjacent spinal levels need to be fused, it becomes necessary to remove the existing implant entirely and replace it with a new one. In some cases, the new one needs to have larger screws that the previous device in order to remain fully attached to the bone. This can cause unnecessary damage to the patient. The necessity for a complete device replacement can also be time consuming and costly, and it can require a long healing period. Some embodiments of the present systems and methods address these challenges by allowing previously installed devices to remain in place during a revision surgery, or by allowing components to be selectively replaced or swapped out without requiring the entire implant to be removed. Thus, many features of the present systems and methods provide advantages over competing devices.

In some embodiments, an additional portion of the coupling assembly 100 can be added to an existing portion (e.g., a second portion 100b can be added onto a first portion 100a) by building off one end (e.g., a first end 122) of a connector 120, without the need for bulky components to extend past (or even to reach) the first portion. For example, as shown in FIG. 2C, no part of the third portion 100c reaches past (or even reaches at all) the second portion 100b, yet the third portion is still able to be joined to the second portion to allow for the coupling assembly to reach an additional level of the patient’s spine.

As another example, in some embodiments, the described systems and methods include a revision spinal fixation system comprising: an existing spinal rod (or any other suitable implant); a collet secured to an end of the existing spinal rod; a coupler comprising a connector receptacle configured to couple with the collet; and an anchor receptacle configured to receive an anchor attached to an adjacent vertebra, wherein the coupler is configured to extend the spinal construct without removal of previously implanted hardware.

As yet another example, some embodiments of the described methods include a method of extending an implanted spinal construct, the method comprising: exposing an end of an implanted rod, attaching a collet, attaching a coupler, and coupling the coupler to a newly implanted anchor without removing an existing tulip assembly.

Any and all of the components in the figures, embodiments, implementations, instances, cases, methods, applications, iterations, instantiations, boxes, and other parts of this disclosure can be combined in any suitable manner. Additionally, any component can be removed, separated from other components, modified with or without modification of like components, or otherwise altered together or separately from anything else disclosed herein.

As used herein, the singular forms “a”, “an”, “the” and other singular references include plural referents, and plural references include the singular, unless the context clearly dictates otherwise. For example, reference to a groove includes reference to one or more grooves, and reference to anchors includes reference to one or more anchors. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object—regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

### ADDITIONAL DESCRIPTION

The features of some embodiments of the systems and methods presented herein may become more apparent from the additional description provided below. In this regard, the description below may include different terminology, examples, features, embodiments, or points of focus than the description above, but again, any of the foregoing can be combined with, substituted for, or otherwise used with all or part of any components of any of the embodiments described above.

In some cases, spinal lumbar fusion surgery can trigger a cascade of adjacent spinal segment degeneration. The levels above and/or below a fused level can become painfully symptomatic within a few years. In some cases, subsequent fusion of an adjacent level can be difficult because the already-fused level still needs hardware to support the relatively weak bone of the fusion mass. However, in some cases, the already-implanted hardware precludes the implantation of new hardware at the adjacent level.

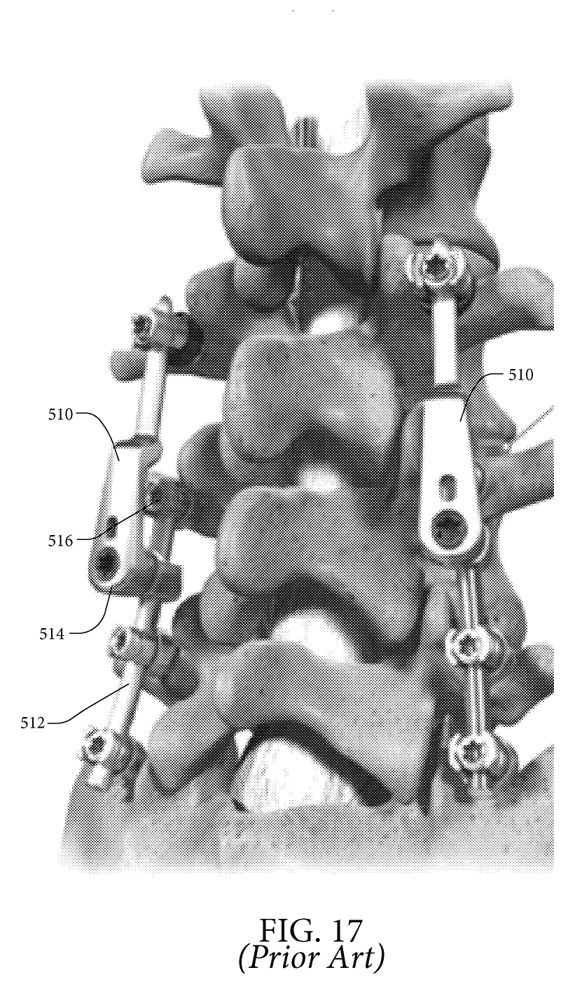

FIG. 17 illustrates an example of a prior-art revision system that uses a relatively large clamping assembly to extend over existing hardware. As discussed below, some embodiments of the present disclosure avoid such bulky structures by coupling directly to an exposed end of an existing construct.

In this regard, removing the already-implanted hardware and then replacing it with a longer construct in accordance with some existing methods can be onerous and risky. So, some have created extending attachments that allow the already implanted hardware to stay in place; but the predicate extending attachments are often very bulky and difficult to use. For example, FIG. 17 illustrates a prior extending attachment 510 adapted to be affixed to a rod 512 of an existing implant assembly via a clamping assembly 514. As may be appreciated from FIG. 17, the clamping assembly 514 must extend posteriorly approximately twice the maximal posterior extension of the existing implant assembly so as to pass over an existing pedicle screw assembly 516 from the existing implant assembly. Accordingly, implantation of the extending attachment 510 can result in a very bulky final assembly at the level of the existing implant adjacent where the existing implant was extended. Additionally, such extending attachments require re-exposing much of the original hardware, which can be painful, time-consuming, and can increase morbidity. Furthermore, in many instances, such implants are used bilaterally, so the problems discussed above are commonly duplicated on each side of the spine during a fixation procedure.

According to some embodiments of the instant systems and methods, systems and methods for extending prior spinal fusion constructs in revision surgery while minimizing surgical exposure of the prior constructs are provided. According to some embodiments, a revision system for extending spinal fusion implants in situ includes a tulip assembly adapted to be fixedly secured to a pedicle screw as part of a first spinal fusion procedure and a construct extension protrusion extending from the tulip assembly in a direction roughly parallel to a next level of a spine adjacent to a level of the spine to be fused as part of the first spinal fusion procedure. The construct extension protrusion may be integrally formed with the tulip assembly.

According to some embodiments, a revision system for extending spinal fusion implants in situ includes a tulip assembly adapted to be fixedly secured to a pedicle screw as part of a second spinal fusion procedure adjacent a site of a prior spinal fusion procedure. The tulip assembly includes a receptacle adapted to be fixedly secured to a construct extension protrusion of a spinal fusion construct from the prior spinal fusion procedure via a press or interference fit.

According to some embodiments, a method for performing spinal fusion to reduce surgical exposure required for any necessary future revision surgery includes assembling in situ a spinal fusion construct adapted to fuse one or more levels of the spine, wherein, upon assembly, a terminal end of the construct includes a construct extension protrusion extending from the construct in a direction roughly parallel to a next level of a spine adjacent to a level of the spine fused as part of a spinal fusion procedure in which the spinal fusion construct is used.

According to some embodiments, a method for performing a spinal fusion procedure while reducing surgical exposure at a site of a previous spinal fusion construct includes exposing a terminal end of the previous spinal fusion construct, the previous spinal fusion construct comprising a construct extension protrusion extending from the construct in a direction roughly parallel to a next level of a spine adjacent to a level of the spine fused as part of a prior spinal fusion procedure, securing a pedicle screw to a next pedicle of the spine, and securing an extension construct between the pedicle screw and the construct extension protrusion, wherein the extension construct is secured to the construct extension protrusion via a press fit or interference fit.

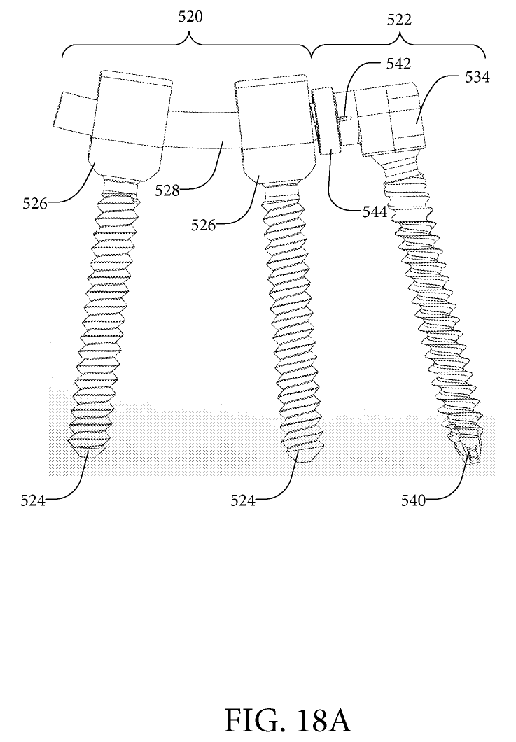

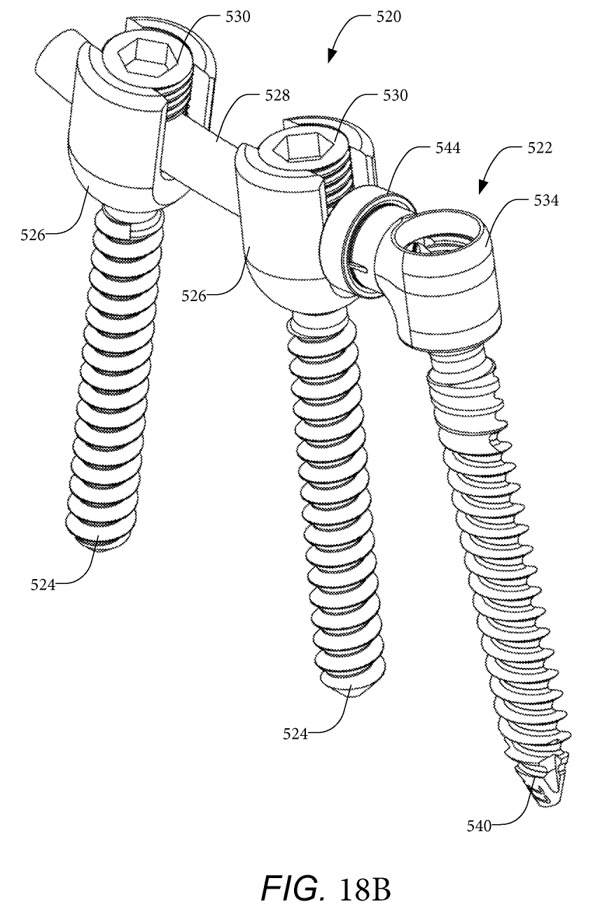

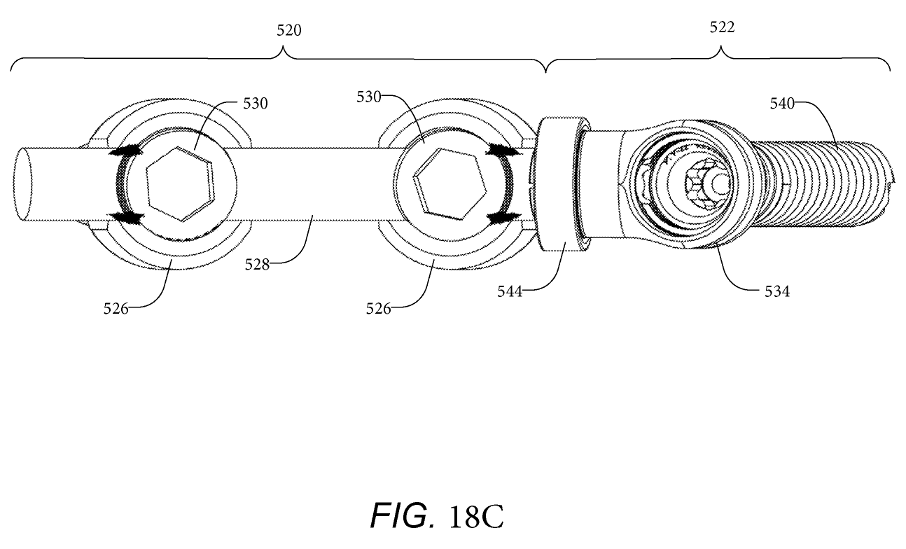

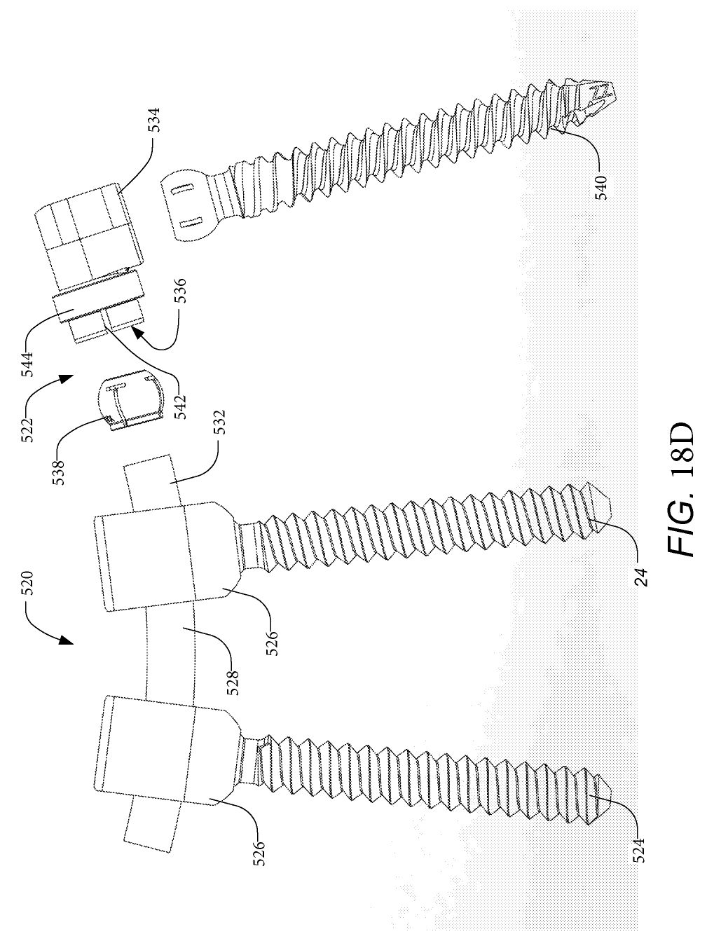

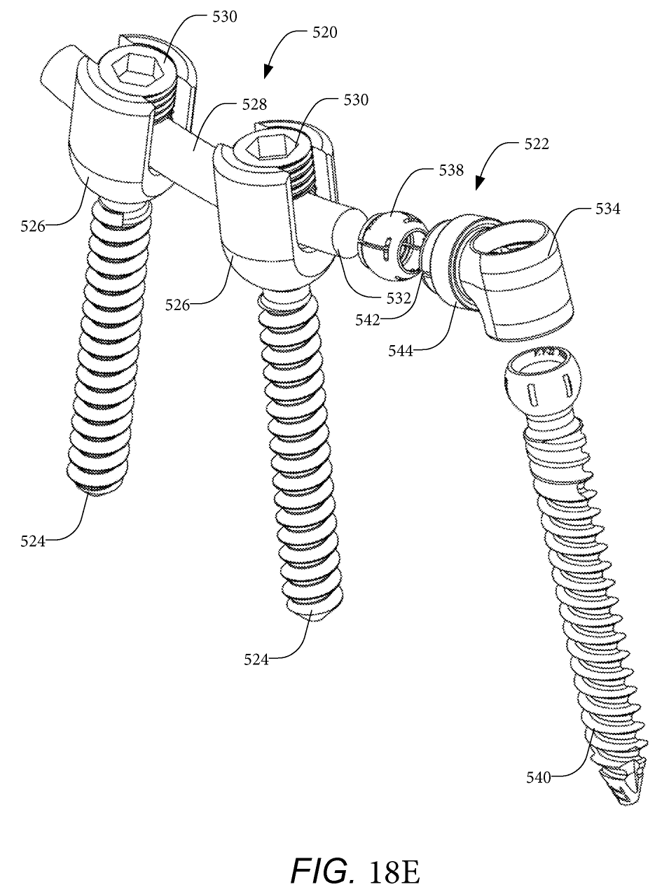

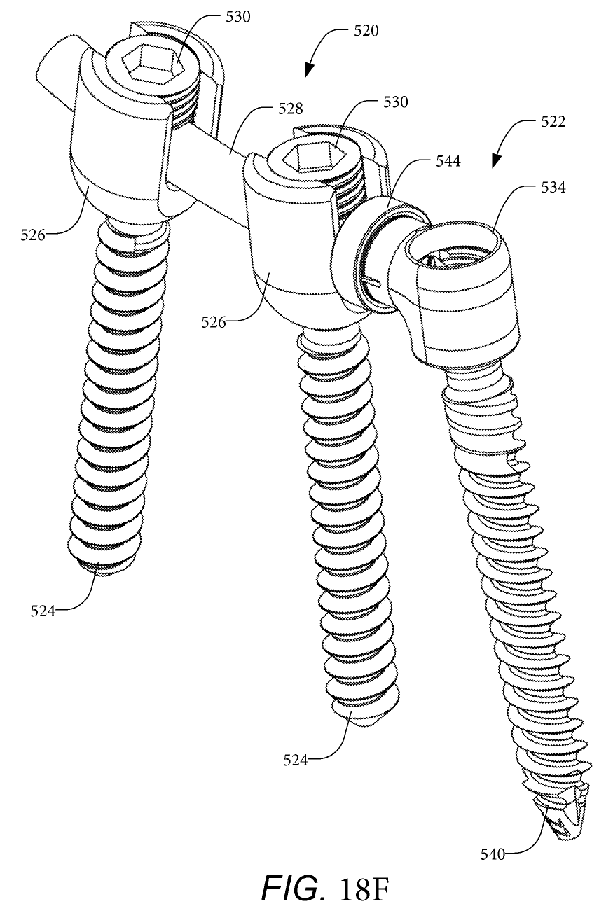

While FIGS. 18A-20 show various views of illustrative embodiments of revision systems embodying some of the principles discussed herein, it should be recognized that the illustrated embodiments are intended to be merely illustrative of the principles discussed herein. Specifically, it should be noted that the various embodiments can be modified to include construct extension protrusions in like manner as is shown in FIGS. 18A-20, and advantages achieved and disclosed in other embodiments can be achieved while also providing the revision advantages provided by the embodiments illustrated in such figures.

FIGS. 18A-18F illustrate various views of a first spinal fusion construct 520 and of a revision spinal fusion construct extender 522. FIG. 18A shows a side view of the first spinal fusion construct 520 assembled with the revision spinal fusion construct extender 522. FIGS. 18C and 18F show view of the first spinal fusion construct 520 assembled with the revision spinal fusion construct 522. FIG. 18D shows a side view of the first spinal fusion construct 520 with an exploded portion of the revision spinal fusion construct 522. FIG. 18E shows a side exploded view of the first spinal fusion construct 520 and the revision spinal fusion construct 522. FIG. 18F shows a perspective view of the first spinal fusion construct 520 assembled with the revision spinal fusion construct 522.

The first spinal fusion construct 520, according to some embodiments, is generally implanted into a patient as part of a spinal fusion procedure for fusing one or more levels of the spine (the embodiments of FIGS. 18A-18F show a first spinal fusion construct 520 for a single level fusion). The first spinal fusion construct of some embodiments includes two pedicle screws 524 that would be implanted into the pedicles of adjacent vertebra (not shown in FIGS. 18A-18F). In some embodiments, a first construct tulip assembly 526 is assembled to each pedicle screw 524, using any suitable methods, including any suitable methods known in the art. Typical devices for affixing tulip assemblies 526 to the pedicle screws 524 can involve a system where the pedicle screw 524 is inserted into the tulip assembly 526 prior to implanting the screw 524, whereupon additional elements of the construct 520 are assembled to the tulip assemblies 526 to fix the position of the tulip assemblies 526 on the pedicle screws 524 and relative to each other. In some cases, the assembly typically depends on the use of a rod 528 extending between tulip assemblies 526, as well as a set screw 530 for each tulip assembly 526. In some cases, the set screw 530 tightens the rod 528 down into the tulip assembly 526, which simultaneously presses the rod 528 down on the top of the pedicle screw 524, locking the entire assembly.

In some embodiments, the rod 528 can be used in conjunction with embodiments of the revision spinal fusion construct 522 to provide a more compact revision fusion to adjacent spinal levels. As discussed above, it is common for fusion of one or more levels of the spine to eventually result in adjacent spinal segment degeneration. The revision spinal fusion construct 522 of some embodiments permits a revision surgery to fuse adjacent spinal segments and add additional fusion hardware without exposing the entire first spinal fusion construct 520, and without requiring bulky additions to the original first spinal fusion construct 520.

Instead, in some embodiments, the revision spinal fusion construct 522 uses a construct extension protrusion 532 of the first spinal fusion construct 520, which may be a protruding portion of the rod 528. The construct extension protrusion may extend in a direction roughly parallel to the next segment or level of the spine, such as within approximately twenty degrees of parallel or within approximately ten degrees of parallel (or at any other suitable angle). This is best seen in the exploded views of FIGS. 18E-18F. In some embodiments, the revision spinal fusion construct 522 includes a revision tulip assembly 534 that includes a receptacle 536 (an example of a female member) adapted to receive the construct extension protrusion 532 therein, either directly or with a slotted ball 538 that is sized and adapted to compress within the receptacle 536 and over the construct extension protrusion 532 as a press or interference fit is created between the construct extension protrusion 532 and the receptacle 536. The revision tulip assembly 534 may include a screw receptacle adapted to receive a revision pedicle screw 540 via a press or interference fit as shown in FIGS. 18A-18F.

In some embodiments, the receptacle 536 may include a slot 542 that permits the receptacle 536 to expand as it receives the construct extension protrusion 532 (and, if present, the slotted ball 538), but to be compressed to fixedly secure the construct extension protrusion 532 (and the slotted ball 538). This compression may be effected by a cinch band 544 adapted to slide back and forth along the exterior of the receptacle 536. While one size of tulip assembly 534 and receptacle 536 is shown in FIGS. 18A-18F, it may be appreciated that the size may be varied so as to vary the spacing between the pedicles screws 524 and the revision pedicle screw 540.

In some embodiments, the surgeon accesses the area of the segment of the spine to be fused, including exposing the construct extension protrusion 532. In this case, however, the surgeon need not expose any remaining portions of the first spinal fusion construct 520. The surgeon then implants the revision pedicle screw 540 using traditional techniques or other techniques. The surgeon then selects an appropriate size of revision tulip assembly 534 to achieve a desired spacing for the new fusion, and, if necessary for the chosen embodiment, a slotted ball 538. The surgeon then places the slotted ball 538 over the construct extension protrusion 532 and locates the revision tulip assembly 534 in the surgical site so that the receptacle 536 receives the slotted ball 538 and construct extension protrusion 532 therein. At this point, there may still be a significant range of motion between the construct extension protrusion 532 and the revision tulip assembly, including polyaxial rotation and flexion and telescopic motion of the construct extension protrusion 532 within the receptacle 536. Then, the surgeon positions the pedicle and the revision tulip assembly 534 appropriately, and creates a press or interference fit between the revision tulip assembly 534 and the construct extension protrusion 532 by sliding the cinch band over the receptacle 536, and creates a press or interference fit between the revision tulip assembly 534 and the revision pedicle screw 540, such as using other methods. Alternately, the order of establishing press or interference fits may be reversed. In any event, when the full press or interference fits are established, the spinal segment is stabilized.

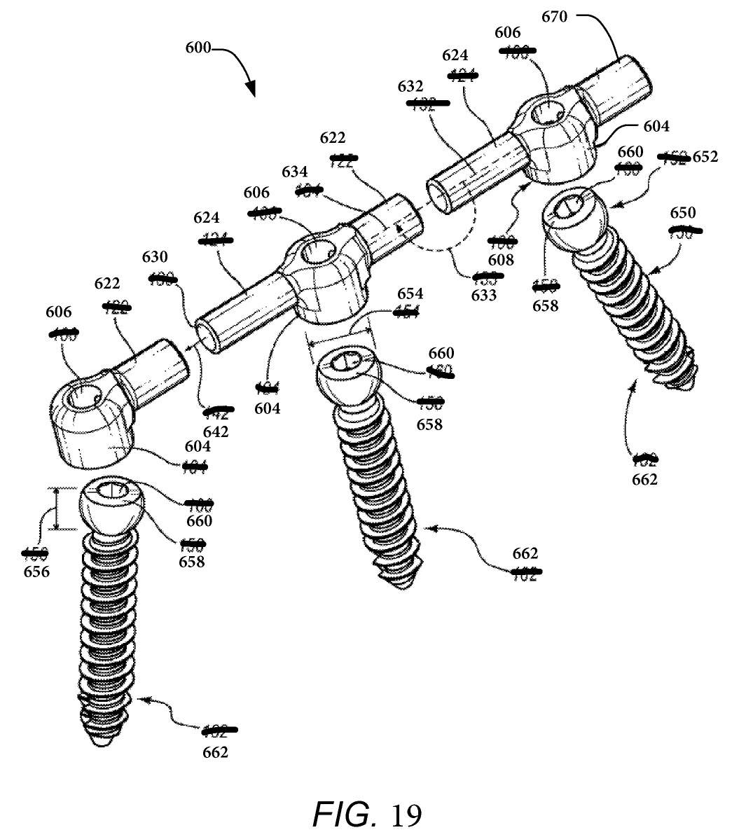

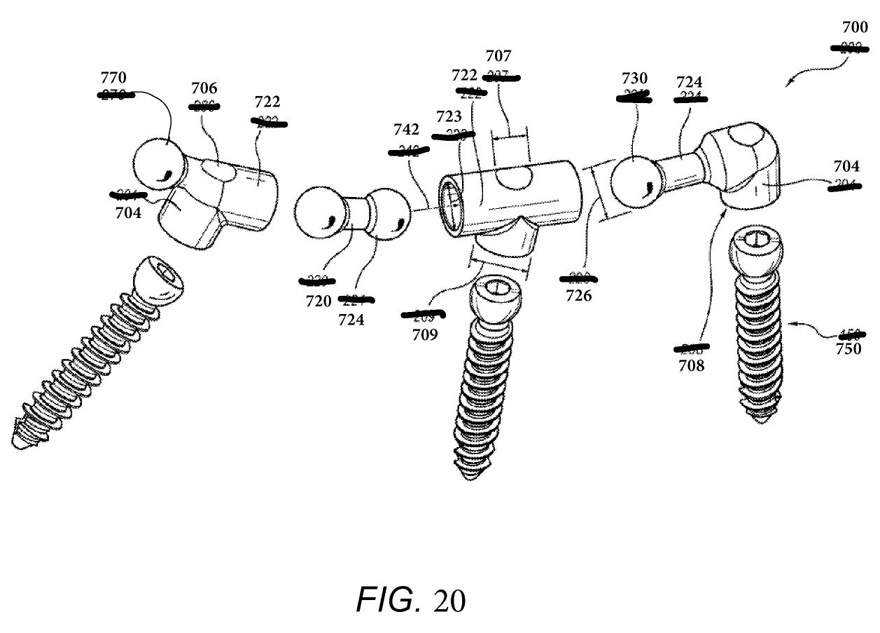

While FIGS. 18A-18F show, according to some embodiments, one style of the first spinal fusion construct 520, it should be appreciated that the construct extension protrusion may be added to essentially any spinal fusion construct, to permit revision procedures in accordance with the disclosure herein. For example, FIG. 19 shows an embodiment of a surgical screw system 600 similar to that shown in FIGS. 1-3 of U.S. Patent No. 9,232,965 that has been modified from that system in that it has a construct extension protrusion 670 added to one end as will be described in more detail below. It should be understood that other embodiments could be similarly modified, as will be illustrated and discussed in more detail with respect to FIG. 20, so the discussion with respect to FIGS. 19-20 is intended to be illustrative only.

In the screw system 600 of FIG. 19, according to some embodiments, various coupling assemblies are illustrated as connecting three surgical screws 650 having a threaded portion 662 together. It will be understood that various embodiments of coupling system are capable of being used with a variety of orthopedic rod placement devices, hooks, and/or surgical screws, including, but not limited to, pedicle screws and orthopedic rods used in spinal surgery. In some cases, the coupling assembly is sometimes referred to as a yolk or tulip assembly.

In some embodiments, the coupling assembly includes at least one body 604; thus, an entire coupling assembly may include a plurality of bodies 604, such as the three bodies 604 illustrated in FIG. 19. The body 604 is illustrated as having a cylindrical shape, although other shapes such as ovoid, spherical, square, and so forth may be used. The body 604 includes an upper bore 606. The body 604 also optionally includes one or more female members 622, one or more male members 624, or a combination of female members 622 and male members 624. Thus, while FIG. 19 illustrates bodies 604 that have one or two male members 624 and/or female members 622, one having skill in the art would understand that a body 604 could have 3, 4, or more male members 624 and/or female members 622, depending on the intended purpose of the coupling assembly. The male member can be rigid, semi-rigid, or flexible, depending on the application.

Referring to FIG. 19, some embodiments of the surgical screw system 600 and the coupling assembly of FIG. 19 are shown in exploded view. The surgical screws 650 of some embodiments include a threaded portion 662 configured to engage the bone of a patient as part of a first orthopedic surgical procedure (e.g. a first spinal fusion encompassing one or more levels). The surgical screws 650 of some embodiments have a head portion 652 that is received by the coupling assembly as will be discussed in further detail below. The head portion 652 of some embodiments has a height 656 and an outer diameter 654. The head portion 652 of some embodiments includes a driving feature 660 for use with various tools to drive the surgical screw 650 into the bone. In some embodiments, as illustrated in FIG. 19, the driving feature 660 includes an Allen-key or hex-head configuration, although other configurations are known in the art, such as hex-a-lobe. The surgical screw 650 of some embodiments includes a screw long axis and a screw cannula. As one having skill in the art would understand, the screw cannula of some embodiments allows the surgical screw to be maneuvered over and to receive a Kirschner wire (typically referred to as a K-wire; not illustrated), a type of guide wire used to aid in the precise placement of the surgical screw in the bone.

In some embodiments, the male member 624 includes a long axis 632 that aligns, to a greater or lesser degree with a first axis 634 of the body 604. By align, it is meant (in this instance) that the long axis 632 and the first axis 634 will be approximately coincident when the male member 624 is inserted via a force 642 into the female member 622 of the body 604. As will be discussed in further detail below, it is often not practical and, sometimes, even possible, to have the long axis 632 and the first axis 634 be coincident due to the tolerances of the manufacturing and surgical placement process, simple variances in the anatomies between patients, and other factors. Thus, there often exists a first angle 633 between the intersection of the long axis 632 and the first axis 634.

In some embodiments, the body 604 optionally includes a cannula having a cannula inner diameter (or height, if the cannula is not cylindrical). The cannula is, in some cases, connected between the female member 622 and the upper bore 606 and provides a route for any fluids, such as blood, entrapped in the female member 622 to escape when the male member 624 is inserted into the female member 622.

The female member 622 of some embodiments has a female inner diameter (or height, if the female member 622 is not cylindrical) and the male member 624 has an outer diameter (or height, if the male member 624 is not cylindrical). The female member 622 is, in some embodiments, configured to receive and to provide an interference fit or press fit to the male member 624. The term interference fit can, in some cases, be interpreted broadly as including the joining of any two mating parts such that one or the other (or both) parts slightly deviate in size from their nominal dimension, thereby deforming each part slightly, each being compressed, the interface between two parts creating a union of extremely high friction. The word interference can refer to the fact that one part slightly interferes with the space that the other is occupying in its nominal dimension. In some embodiments, the interference fit can be configured to require at least 800 pounds of force to remove the male member 624 from the female member 622.

In some embodiments, the female inner diameter 623 can be constant, or it can be sloped or have steps through the inner length of the female member 622 up until a shoulder that prevents the male member 624 from advancing any further into the female member 622. Thus, the female inner diameter in some cases may be larger than an interference fit diameter near a mouth of the female member 622, thereby allowing the male member 624 to rotate, pivot, enter, and exit freely or semi-freely within the female member 622. As the male member 624 is (in some cases) pressed further into the female member 622, the female inner diameter optionally necks down, becoming smaller or narrower, either smoothly or in steps, until an interference fit is achieved. Thus, it will be understood that the female member 622 optionally provides, in some cases, the full or partial ability to adjust the distance between bodies 604.

In some cases, the shoulder optionally narrows or necks down from the female inner diameter to the cannula inner diameter, or it can optionally neck down in steps. In some embodiments, the shoulder optionally includes a lip or ridge that prevents a male face 630 of the male member 624 from advancing beyond the shoulder.

The body or bodies 604, in some embodiments, include an upper bore 606 having a bore inner diameter (or width, if the upper bore 606 is not cylindrical). In some cases, the upper bore 606 is fluidly connected to the cannula. The upper bore 606 of some embodiments is also fluidly connected to a receptacle 608. In some cases, the bore inner diameter is less than the receptacle 608 inner diameter (which can include a diameter of a circle or an inner maximum width if the receptacle 608 is not cylindrical). In some cases, the bore inner diameter 607 is sufficiently wide to accommodate a K-wire or other guiding mechanism threaded through the screw cannula as discussed above.

The receptacle 608 of some embodiments, in addition to having a receptacle inner diameter, has a height and a shoulder, or screw head contact surface. As with the female member 622, the receptacle 608 of some embodiments can be configured to receive a head portion 652 of the surgical screw 650 and to provide an interference or press fit between the receptacle 608 and the head portion 652. The receptacle inner diameter can be constant, or it can be sloped or have steps though the inner height of the receptacle 608 up until the screw head contact surface or shoulder that prevents the head portion 652 from advancing any further into the receptacle 608. Thus, the receptacle inner diameter may be larger than an interference-fit diameter near a mouth of the receptacle 608, thereby allowing the head portion 652 to rotate, enter, and exit freely or semi-freely within the receptacle 608. As the head portion 652 is pressed further into the receptacle 608, the receptacle inner diameter of some embodiments necks down, becoming smaller or narrower, either smoothly or in steps (or otherwise), until an interference fit is achieved.

The body 604 and, hence, the receptacle 608 of some embodiments includes a second axis. The second axis may align, to a greater or lesser degree with the screw long axis of the surgical screw 650, as is the case for the middle body 604 in FIG. 19. In some cases, the screw long axis and the second axis are approximately coincident when the head portion 652 of the surgical screw 650 is inserted into the receptacle 608 of the body 604. As will be discussed in further detail below, it is often not practical and, sometimes, even possible (with some configurations), to have the screw long axis and the second axis be coincident due to the tolerances of the manufacturing and surgical placement process, simple variances in the anatomies between patients and other factors. In some embodiments, the screw long axis intersects the second axis of the body at a second angle, as illustrated in the leftmost body 604 of FIG. 19.

It should be noted that some embodiments, such as some embodiments similar to that shown in FIG. 19, differ from the devices of FIGS. 1-3 of U.S. Patent No. 9,232,965 in that they include a construct extension protrusion 670 extending from the last or rightmost body 604 shown in FIG. 19. The construct extension protrusion 670 in some embodiments is approximately cylindrical, and it may have any desired diameter, though the diameter of the construct extension protrusion 670 of some embodiments is consistent along the length of the construct extension protrusion 670 in this type of example. The construct extension protrusion 670 of this example is adapted to receive a slotted ball 538 as discussed above. While not shown in FIG. 19, an additional construct extension protrusion 670 could similarly be provided to the leftmost body 604 of FIG. 19, allowing for a later revision procedure at that end of the surgical screw system 600 as well.

FIG. 20 shows an embodiment of a surgical screw system 700 similar to that shown in FIGS. 4-6 of U.S. Patent No. 9,232,965 that has been modified from that system in that it has a construct extension protrusion 770 added to one end as will be described in more detail below. It should be understood that other embodiments could be similarly modified, so the discussion with respect to FIG. 20 (as was with FIG. 19) is intended to be illustrative only, and one of ordinary skill in the art will readily appreciate from the discussion of these two figures. how embodiments of systems from any of the related patents and applications incorporated herein (including at least U.S. Serial No. 11/952,709, U.S. Patent No. 9,232,965, U.S. Serial No. 13/455,854, and U.S. Serial No. 14/060,757) could be modified to incorporate one or more construct extension protrusions 670 or 770 as discussed herein.

Accordingly, FIG. 20 illustrates another embodiment of a surgical screw system 700 that includes a plurality of another embodiment of coupling assemblies illustrated as connecting three surgical screws 750 having a threaded portion together. The surgical screws 750 illustrated were previously described above, thus reference should be made to that description.

In some embodiments, the coupling assembly includes at least one body 704; thus, an entire coupling assembly of some embodiments may include a plurality of bodies 704, such as the three bodies 704 illustrated in FIG. 20. The body 704 is illustrated as having a cylindrical shape, although other shapes such as ovoid, spherical, square, and so forth fall within the scope of the disclosure. The body 704 of some embodiments includes an upper bore 706. The body 704 of some embodiments also optionally includes one or more female members 722, one or more male members 724, a combination of female members 722 and male members 724, and, optionally, a coupling rod 720 that includes two or more male members 724 or female members 722. (Illustrated is coupling rod 720 with male members 724, but this is just one embodiment of the coupling rod, as will be understood). Thus, while FIG. 20 illustrates bodies 704 that have one or two male members 724 and/or female members 722, and one coupling rod 720, one having skill in the art would understand that a body 704 could have 3, 4, or more male members 724 and/or female members 722 and/or coupling rods 720, depending on the intended purpose of the coupling assembly.

As with some embodiments as shown in FIG. 19, the male member 724 and the coupling rod 720 can include a long axis that aligns, to a greater or lesser degree with a first axis of the body. In some cases, the long axis and the first axis will be approximately coincident when the male member 724 (whether included with a body 704 or a coupling rod 720) is inserted via a force 742 into the female member 722 of the body 704. As will be discussed in further detail below, it is often not practical and, sometimes, even possible, to have the long axis and the first axis be coincident due to the tolerances of the manufacturing and surgical placement process, simple variances in the anatomies between patients and other factors. Thus, there often exists a first angle 733 between the intersection of the long axis and the first axis. Some embodiments optionally include a male member 724 that has a male face 730 that is a sphere in this instance, although other shapes and configurations, including ovoid, square, and the like, fall within the scope of the disclosure. An advantage of the male face 730 of spherical shape (in some cases) is that it provides for a greater range of motion and possible angles 733 than may otherwise be accommodated, thus providing greater ease of use and accommodation for a wider variety of factors that a surgeon might encounter while connecting the coupling assembly to a surgical screw or screws.

The body 704 of some embodiments includes a cannula having a cannula inner diameter (or height, if the cannula is not cylindrical). The cannula is connected between the female member 722 and the upper bore 706 and provides a route for any fluids, such as blood, entrapped in the female member 722 to escape when the male member 724 is inserted into the female member 722.

The female member 722 of some embodiments has a female inner diameter 723 and the male member 724 includes the male face 730 that has an outer diameter 726. The female member 722 is configured to receive and to provide an interference fit or press fit to the male face 730 of the male member 724.

The female inner diameter 723 can be constant, or it can be sloped or have steps though the inner length of the female member 722 up until a shoulder that prevents the male face 730 of the male member 724 from advancing any further into the female member 722. Thus, the female inner diameter 723 may be larger than an interference fit diameter near a mouth of the female member 722, thereby allowing the male face 730 of the male member 724 to rotate, enter, and exit freely or semi-freely within the female member 722. As the male member 724 is pressed further into the female member 722, the female inner diameter 723 optionally necks down, becoming smaller or narrower, either smoothly or in steps, until an interference fit is achieved. Thus, it will be understood that the female member 722 optionally provides, in part, the ability to adjust the distance between bodies 704.

The shoulder optionally narrows or necks down from the female inner diameter 723 to the cannula inner diameter, or it can optionally neck down in steps. The shoulder optionally includes a lip or ridge that prevents the male face 730 of the male member 724 from advancing beyond the shoulder.

The body or bodies 704, in some embodiments, include an upper bore 706 having a bore inner diameter 707. As noted, the upper bore 706 is fluidly connected to the cannula. The upper bore 706 of some embodiments is also fluidly connected to a receptacle 708 that receives the screw head. The bore inner diameter 707 of some embodiments is less than the receptacle inner diameter (or width, if the receptacle is not cylindrical). Optionally, the bore inner diameter 707 is sufficiently wide to accommodate a K-wire or other guiding mechanism threaded through the screw cannula as discussed above.

In some embodiments, the receptacle 708, in addition to the receptacle inner diameter, has a height and a shoulder, or screw head contact surface. As with the female member 722, the receptacle 708 (in some cases) is configured to receive a head portion 752 of the surgical screw 750 and to provide an interference or press fit between the receptacle 708 and the head portion 752. The receptacle inner diameter can be constant, or it can be sloped or have steps though the inner height of the receptacle 708 up until the screw head contact surface or shoulder that prevents the head portion 752 from advancing any further into the receptacle 708. Thus, the receptacle inner diameter of some embodiments may be larger than an interference-fit diameter near a mouth of the receptacle 708, thereby allowing the head portion 752 to rotate, pivot, enter, and exit freely or semi-freely within the receptacle 708. As the head portion 752 is pressed further into the receptacle 708, the receptacle inner diameter optionally necks down, becoming smaller or narrower, either smoothly or in steps, until an interference fit is achieved.

In some embodiments, the body 704 and, hence, the receptacle 708 includes a second axis. The second axis may align, to a greater or lesser degree with the screw long axis of the surgical screw 750, as is the case for the middle body 704 in some embodiments as shown in FIG. 20.

Some embodiments, as shown in FIG. 20, differ from the embodiments of FIGS. 4-6 of U.S. Patent No. 9,232,965 in that they include the construct extension protrusion 770 extending from the last or leftmost body 704 shown in FIG. 20. The construct extension protrusion 770 of some embodiments includes an approximately spherical portion, and it may have any desired diameter. The construct extension protrusion 770 of some embodiments is not used with a slotted ball 538, as the spherical portion is already present. While not shown in FIG. 20, an additional construct extension protrusion 770 could similarly be provided to the rightmost body 704 of FIG. 20, allowing for a later revision procedure at that end of the surgical screw system 700 as well.

The construct extension protrusion 670 of FIG. 19 and the construct extension protrusion 770 of FIG. 20 illustrate that such construct extension protrusions 670, 770 may be unitarily formed with their respective bodies 604, 704. Accordingly, it may be said that construct extension protrusions of some embodiments may be unitarily formed with tulip assemblies such as any tulip assembly or coupling assembly disclosed in the related patents and applications that have been incorporated herein by reference.

Some embodiments of the present systems and methods provide a simple-to-use and low-profile construct extender for revision surgery. In some cases, it attaches to the existing hardware at the existing rod end.

In some embodiments, the required surgical exposure is reduced because access can be contained within the to-be-fused segment(s).

In some embodiments, the novel extender provides a stronger, more reliable connection.

In some embodiments, the unique method of locking provides a lower bulk implant.

In some embodiments, the spherical ball can connect the new hardware to the existing hardware. This connection can be polyaxial and can be telescopic. Locking the new hardware can make rigid the mate to the existing hardware.

In some embodiments, the connection between new and old does not have to employ a spherical ball. An interference fit about the old rod can be accomplished with other geometries and with fewer components. The locking ring and spherical ball could be eliminated and the new hardware could simply be pressed onto the end of the existing rod to create an interference fit mate between the old and the new. However, the spherical ball and the locking ring simplify the sizing of the new construct.

What is claimed is:

1. A coupling assembly for use in a spinal fusion procedure, the coupling assembly comprising:
an anchor configured to couple to a spinal vertebra, the anchor comprising:
a shaft configured to be embedded in the spinal vertebra; and
a head;
a coupler comprising:
an anchor receptacle comprising:
an opening formed in a bottom of the anchor receptacle; and
a retention groove extending into an inner surface of the anchor receptacle, the anchor receptacle being configured to receive the head of the anchor through the opening and retain the head of the anchor within the anchor receptacle;
a connector portion configured to span a distance between the anchor and an additional vertebra when the anchor is coupled to the spinal vertebra;
a collet configured to couple to a connector; and
a connector receptacle configured to couple to the collet while the collet is coupled to the connector.

2. The coupling assembly of claim 1, wherein the head of the anchor includes a recess configured to receive a wand, and wherein the head of the anchor further comprises a drive formed within the recess.

3. The coupling assembly of claim 2, wherein the anchor receptacle has a top with an additional opening formed therein, the additional opening being configured to receive the wand therethrough.

4. The coupling assembly of claim 1, wherein the retention groove extends around a perimeter of the inner surface of the anchor receptacle.

5. The coupling assembly of claim 4, wherein the retention groove extends around an entirety of the perimeter of the inner surface of the anchor receptacle.

6. The coupling assembly of claim 1, wherein the retention groove comprises an edge configured to press against a curved surface of the head of the anchor.

7. The coupling assembly of claim 1, wherein the retention groove has a polygonal cross-sectional shape.

8. The coupling assembly of claim 1, wherein the retention groove is disposed in a lower third of the anchor receptacle.

9. The coupling assembly of claim 1, wherein the shaft of the anchor comprises a hook.

10. The coupling assembly of claim 1, wherein the shaft of the anchor comprises a threading.

11. A kit for providing a coupling assembly for use in connection with a spinal fusion procedure, the kit comprising:
a first anchor configured to couple to a spinal vertebra, the anchor comprising:
a shaft configured to be embedded in the spinal vertebra; and
a head;
a first coupler comprising:
an anchor receptacle comprising:
an opening formed in a bottom of the anchor receptacle; and
a retention groove extending into an inner surface of the anchor receptacle, the anchor receptacle being configured to receive the head of the anchor through the opening and retain the head of the anchor within the anchor receptacle;
a connector portion configured to span a distance between the anchor and an additional vertebra when the anchor is coupled to the spinal vertebra;
a first collet; and
a connector receptacle configured to couple to the first collet.

12. The kit of claim 11, further comprising a second collet, wherein the first collet has a first size, and wherein the second collet has a second size, and wherein the connector receptacle is configured to selectively couple to each of the first collet and the second collet.

13. The kit of claim 11, further comprising a second coupler, wherein the first coupler has a first size and the second coupler has a second size, and wherein the first coupler and the second coupler are interchangeable in forming the coupling assembly.

14. The kit of claim 11, further comprising a second anchor, wherein the first anchor has a first size and the second anchor has a second size, and wherein each of the first anchor and the second anchor is configured to selectively couple to the anchor receptacle of the first coupler.

15. The kit of claim 11, further comprising a wand configured to be inserted through the anchor receptacle of the first coupler.

16. The kit of claim 11, further comprising an anchor locker configured to press the first coupler and the first anchor together to cause them to couple together.

17. The kit of claim 11, further comprising a crimper.

18. A method for providing a coupling assembly for use in a spinal fusion procedure, the method comprising:
preparing an existing portion of an implant;
selecting a collet for a new portion of the implant;
installing the collet on the existing portion of the implant;
installing an anchor for the new portion of the implant;
selecting a coupler for the new portion of the implant;
installing the coupler for the new portion of the implant; and
locking the coupler, thereby coupling the new portion of the implant to the existing portion of the implant.

19. The method of claim 18, wherein the preparing the existing portion of the implant comprises removing bone growth around an end of a connector of the existing implant.

20. The method of claim 19, wherein the installing the collet on the existing portion of the implant comprises:
sliding the collet onto the end of the connector; and
deforming at least one of the collet and the connector to prevent the collet from sliding off the end of the connector.

21. The method of claim 18, wherein the locking the coupler comprises shifting a locking element from an unlocked position to a locked position.

22. The method of claim 18, further comprising pressing an anchor receptacle of the coupler onto a head of the anchor.

23. A coupling assembly for use in a spinal fusion procedure, the coupling assembly comprising:
a collet that is configured to (i) be slid onto an end of a first rod that is coupled to a spine of a patient and (ii) be fixedly coupled to the rod;
a coupler comprising a second rod; and
a set screw that is configured to extend into the coupler to couple the coupler at a polyaxial angle to the end of the first rod.

24. A coupling assembly for use in a spinal fusion procedure, the coupling assembly comprising:
a collet configured to slide onto an end of a connector; and
a coupler comprising:
a connector receptacle configured to receive the collet while the collet is disposed on the connector; and
a locking element configured to lock the collet in the connector receptacle, thereby securing the connector to the coupler.

25. The coupling assembly of claim 24, wherein the locking element comprises a set screw.

26. The coupling assembly of claim 24, wherein the locking element comprises a locking ring.

27. The coupling assembly of claim 24, wherein the collet comprises a neck configured to couple to the end of the connector such that a body of the collet extends past the end of the connector while the collet is coupled to the connector.
